(19) United States
(12) Patent Application Publication
(10) Pub. No.: US 2005/0033232 A1
(43) Pub. Date: Feb. 10, 2005

(54) Infusion apparatus with modulated flow control

(76) Inventor: Marshall S. Kriesel, St. Paul, MN (US)

Correspondence Address: James E. Brunton;Suite 860, 700 North Brand Blvd., P. O. Box 29000, Glendale, CA, 91203, US

(21) Appl. No.: 10/634,624

(22) Filed: Aug. 5, 2003

(51) Int. Cl. A61M 37/00

(52) U.S. Cl. 604/131; 417/472

(57) ABSTRACT

A compact fluid dispenser for use in controllably dispensing fluid medicaments, such as antibiotics, oncolytics, hormones, steroids, blood clotting agents, analgesics, and like medicinal agents from prefilled containers at a uniform rate. The dispenser uniquely includes a stored energy source that is provided in the form of a substantially constant-force, compressible-expandable wave spring that provides the force necessary to continuously and uniformly expel fluid from the device reservoir. The device further includes a fluid flow control assembly that precisely controls the flow of medicament solution to the patient. Additionally, the device includes a novel modulating assembly for controllably modulating the force exerted by the wave spring tending to expel the fluid from the device reservoir.

### BACKGROUND OF THE INVENTION

1. Field of the Invention

The present invention relates generally to medicament infusion devices. More particularly, the invention concerns an improved apparatus for infusing medicinal agents into an ambulatory patient at specific rates over extended periods of time, which apparatus includes a novel modulated energy source provided in the form of a compressible spring, and a novel flow rate control means for precisely controlling the rate of fluid flow from the reservoir of the device.

2. Discussion of the Prior Art

A number of different types of medicament dispensers for dispensing medicaments to ambulatory patients have been suggested. Many of the devices seek either to improve or to replace the traditional gravity flow and hypodermic syringe methods, which have been the standard for delivery of liquid medicaments for many years.

The prior art gravity flow methods typically involve the use of intravenous administration sets and the familiar flexible solution bag suspended above the patient. Such gravametric methods are cumbersome, imprecise and require bed confinement of the patient. Periodic monitoring of the apparatus by the nurse or doctor is required to detect malfunctions of the infusion apparatus.

Many medicinal agents require an intravenous route for administration thus bypassing the digestive system and precluding degradation by the catalytic enzymes in the digestive tract and the liver. The use of more potent medications at elevated concentrations has also increased the need for accuracy in controlling the delivery of such drugs. The delivery device, while not an active pharmacologic agent, may enhance the activity of the drug by mediating its therapeutic effectiveness. Certain classes of new pharmacologic agents possess a very narrow range of therapeutic effectiveness, for instance, too small a dose results in no effect, while too great a dose can result in a toxic reaction.

For those patients that require frequent injections of the same or different amounts of medicament, the use of the hypodermic syringe method of delivery is common. However for each injection, it is necessary to first draw the injection dose into the syringe, then check the dose and, after making certain that all air has been expelled from the syringe, finally, inject the dose either under bolus or slow push protocol. This cumbersome and tedious procedure creates an unacceptable probability of debilitating complications, particularly for the elderly and the infirm.

As will be appreciated from the discussion, which follows, the apparatus of the present invention is uniquely suited to provide precise, continuous fluid delivery management at a low cost in those cases where a variety of precise dosage schemes are of utmost importance. An important aspect of the apparatus of the present invention is the provision of novel fill means for filling the reservoir of the device using a conventional medicament vials or cartridge containers of various types having a pierceable septum. Another novel feature of the apparatus of the present invention comprises a unique, modulated stored energy source. A further unique feature is the provision of various fluid flow rate control means, including an embedded micro fluidic capillary multichannel flow rate control means, which enables precise control of the rate of fluid flow of the medicament to the patient. More particularly, the apparatus of the present invention includes a unique, adjustable fluid flow rate mechanism, which enables the fluid contained within the reservoir of the device to be precisely dispensed at various selected rates.

The apparatus of the present invention can be used with minimal professional assistance in an alternate health care environment, such as the home. By way of example, devices of the invention can be comfortably and conveniently removably affixed to the patient's body or clothing and can be used for the continuous infusion of injectable anti-infectives, hormones, steroids, blood clotting agents, analgesics, and like medicinal agents. Similarly, the devices of the invention can be used for most I-V chemotherapy and can accurately deliver fluids to the patient in precisely the correct quantities and at extended microfusion rates over time.

By way of summary, the apparatus of the present invention uniquely overcomes the drawbacks of the prior art by providing a novel, disposable dispenser of simple but highly reliable construction. A particularly important aspect of the apparatus of the present invention resides in the provision of a novel, self-contained modulated energy source comprising a compressible-expandable spring members that provides the modulated force necessary to uniformly and precisely dispense various solutions from standard prefilled vial containers that can be conveniently loaded into the apparatus. Because of the simplicity of construction of the apparatus of the invention, and the unique nature of the energy source, the apparatus can be manufactured at low cost without in any way sacrificing accuracy and reliability.

With regard to the prior art, one of the most versatile and unique fluid delivery apparatus developed in recent years is that developed by the present inventor and described in U.S. Pat. No. 5,205,820. The components of this novel fluid delivery apparatus generally include: a base assembly, an elastomeric membrane serving as a stored energy means, fluid flow channels for filling and delivery, flow control means, a cover, and an ullage which comprises a part of the base assembly.

Another prior art patent issued to the present applicant, namely U.S. Pat. No. 5,743,879, discloses an injectable medicament dispenser for use in controllably dispensing fluid medicaments such as insulin, anti-infectives, analgesics, oncolylotics, cardiac drugs biopharmaceuticals, and the like from a prefilled container at a uniform rate. The dispenser, which is quite dissimilar in construction and operation from that of the present invention, includes a stored energy source in the form of a compressively deformable, polymeric elastomeric member that provides the force necessary to controllably discharge the medicament from a prefilled container, which is housed within the body of the device. After having been deformed, the polymeric, elastomeric member will return to its starting configuration in a highly predictable manner.

Another important prior art fluid delivery device is described in the U.S. Pat. No. 6,063,059 also issued to the present inventor. This device, while being of a completely different construction embodies a compressible-expandable stored energy source somewhat similar to that used in the apparatus of the present invention.

Still another prior art fluid delivery device, in which the present inventor is also named as an inventor, is described in U.S. Pat. No. 6,086,561. This latter patent incorporates a fill system that makes use of conventional vials and cartridge medicament containers.

### SUMMARY OF THE INVENTION

It is an object of the present invention to provide a compact fluid dispenser for use in controllably dispensing fluid medicaments, such as, antibiotics, oncolytics, hormones, steroids, blood clotting agents, analgesics, and like medicinal agents from prefilled containers at a uniform rate.

Another object of the invention is to provide a small, compact fluid dispenser that includes a housing to which fill vials can be connected for filling the dispenser reservoir with the fluid.

Another object of the invention is to provide a dispenser in which a stored energy source is provided in the form of a compressible-expandable wave spring that provides the force necessary to continuously and uniformly expel fluid from the device reservoir.

Another object of the invention is to provide a dispenser of the class described, which includes novel modulating means for modulating the force exerted by the compressible-expandable wave spring.

Another object of the invention is to provide a dispenser as described in the preceding paragraphs that includes a novel fluid flow control assembly that precisely controls the flow of the medicament solution to the patient.

Another object of the invention is to provide a dispenser that includes precise variable flow rate selection.

Another object of the invention is to provide a fluid dispenser, which is adapted to be used with conventional prefilled drug containers to deliver beneficial agents there from in a precise and sterile manner.

Another object of the invention is to provide a fluid dispenser of the class described which is compact, lightweight, is easy for ambulatory patients to use, is fully disposable, and is extremely accurate so as to enable the continuous infusion of precise volumes of medicament over prescribed periods of time.

Another object of the invention is to provide a device of the character described which embodies a novel fluid volume indicator that provides a readily discernible visual indication of the volume of fluid remaining in the device reservoir.

Another object of the invention is to provide a self-contained medicament dispenser which is of very simple construction and yet extremely reliable in use.

Another object of the invention is to provide a fluid dispenser as described in the preceding paragraphs, which is easy and inexpensive to manufacture in large quantities.

### BRIEF DESCRIPTION OF THE DRAWINGS

FIG. 1 is a generally perspective front view of one embodiment of the medicament infusion apparatus of the present invention for dispensing fluids at a uniform rate.

FIG. 2 is an enlarged, longitudinal cross-sectional view of the apparatus shown in FIG. 1.

FIG. 2A is an enlarged, fragmentary, cross-sectional view of a portion of the collapsible bellows component of the apparatus shown in FIG. 1.

FIG. 3 is a cross-sectional view taken all lines 3-3 of FIG. 2.

FIG. 4 is a cross-sectional view taken along lines 4-4 of FIG. 2.

FIG. 5 is a cross-sectionalview taken along lines 5-5 of FIG. 2.

FIG. 6 is a left end view of the apparatus shown in FIG. 2.

FIG. 7 is a cross-sectional view taken along lines 7-7 of FIG. 2.

FIG. 8 is an interior view of the bezel component of the apparatus shown in FIG. 2.

FIG. 9 is a cross-sectional view taken along lines 9-9 of FIG. 8.

FIG. 10 is a generally perspective, exploded view of the apparatus of the invention shown in FIG. 2.

FIG. 10A is an enlarged, generally perspective, exploded rear view of the forward portion of the apparatus shown in FIG. 10.

FIG. 11 is an enlarged fragmentary cross-sectional view of a portion of the device housing showing one form of the air collar and control shaft of the stored energy means of the invention.

FIG. 12 is a cross-sectional view taken along lines 12-12 of FIG. 11.

FIG. 13 is an enlarged fragmentary cross-sectional view similar to FIG. 11, but showing the control shaft of the stored energy means moved into a second position.

FIG. 14 is a cross-sectional view taken along lines 14-14 of FIG. 13.

FIG. 15 is an enlarged fragmentary cross-sectional view showing the stored energy means of the apparatus of the invention in an intermediate fluid delivery position.

FIG. 16 is an enlarged fragmentary cross-sectional view similar to FIG. 15, but illustrating the position of the operating components following completion of the delivery of the medicinal fluid from the fluid reservoir of the device.

FIG. 16A is a generally diagrammatic, graphical view illustrating the manner in which the force generated by the wave spring loading is modulated by the compression modulator or modulating means of the invention FIG. 17 is a generally perspective, front view of one form of the fluid flow control assembly of the apparatus of the invention.

FIG. 17A is a generally perspective, exploded front view of the fluid flow control assembly shown in FIG. 17.

FIG. 18 is a greatly enlarged, fragmentary cross-sectional view of one of the flow control channels formed in the flow control member shown in the central portion of FIG. 17.

FIG. 19 is a generally perspective, rear view of the fluid flow control assembly of the apparatus of the invention.

FIG. 20 is a generally perspective, exploded rear view of the fluid flow control assembly shown in FIG. 19.

FIG. 21 is a generally perspective view of an alternate form of the flow control member of the invention.

FIG. 21A is a generally perspective view of yet another form of the flow control member of the invention.

FIG. 22 is a front view of the assembly shown in FIG. 19.

FIG. 23 is a cross-sectional view taken along lines 23-23 of FIG. 22.

FIG. 24 is a view taken along lines 24-24 of FIG. 23.

FIG. 25 is a cross-sectional view taken along lines 25-25 of FIG. 23.

FIG. 26 is a cross-sectional view taken along lines 26-26 of FIG. 23.

FIG. 27 is a generally perspective front view of an alternate embodiment of the medicament infusion apparatus of the present invention for dispensing fluids at a uniform rate.

FIG. 28 is an enlarged, longitudinal cross-sectional view of the apparatus shown in FIG. 27.

FIG. 29 is a left end a view of the alternate embodiment of the invention shown in FIG. 27.

FIG. 30 is a right end view of the alternate embodiment of the invention shown in FIG. 27.

FIG. 31 is a cross-sectional view taken along lines 31-31 of FIG. 28.

FIG. 32 is a cross-sectional view taken along lines 32-32 of FIG. 28.

FIG. 33 is a cross-sectional view taken along lines 33-33 of FIG. 28.

FIG. 34 is a generally perspective, exploded view of the apparatus of the invention shown in FIG. 28.

FIG. 35 is a fragmentary, cross-sectional view of a portion of the device housing showing the air collar and control shaft of the stored energy means of this latest form of the invention.

FIG. 36 is an enlarged cross-sectional view taken along lines 36-36 of FIG. 35.

FIG. 37 is a fragmentary cross-sectional view similar to FIG. 35, but showing the control shaft of the stored energy means moved into a second position.

FIG. 38 is a cross-sectional view taken along lines 38-38 of FIG. 37.

FIG. 39 is an enlarged fragmentary cross-sectional view showing the stored energy means of this latest form of the apparatus of the invention following completion of the fluid delivery step.

FIG. 40 is an enlarged fragmentary cross-sectional view similar to FIG. 39, but showing the stored energy means of this latest form of the invention in an intermediate fluid delivery position.

FIG. 41 is an enlarged, fragmentary cross-sectional view of the upper right hand portion of the apparatus shown in FIG. 28, better illustrating an alternate form of rate control assembly of the apparatus of this latest form of the invention.

FIG. 42 is a generally perspective fragmentary, exploded view of the upper right hand portion of the apparatus shown in FIG. 27.

FIG. 43 is a greatly enlarged, bottom perspective, exploded view of the rate control assembly of the apparatus of this latest form of the invention.

FIG. 44 is a greatly enlarged, top perspective, exploded view of the rate control assembly of the apparatus of this latest form of the invention.

FIG. 45 is a generally diagrammatic, tabular view illustrating and describing the various types of springs that can be used as the stored energy source of the invention.

FIG. 46 is a generally diagrammatic, tubular view further illustrating and describing the various types of springs that can be used as the stored energy source of the invention.

### DESCRIPTION OF THE INVENTION

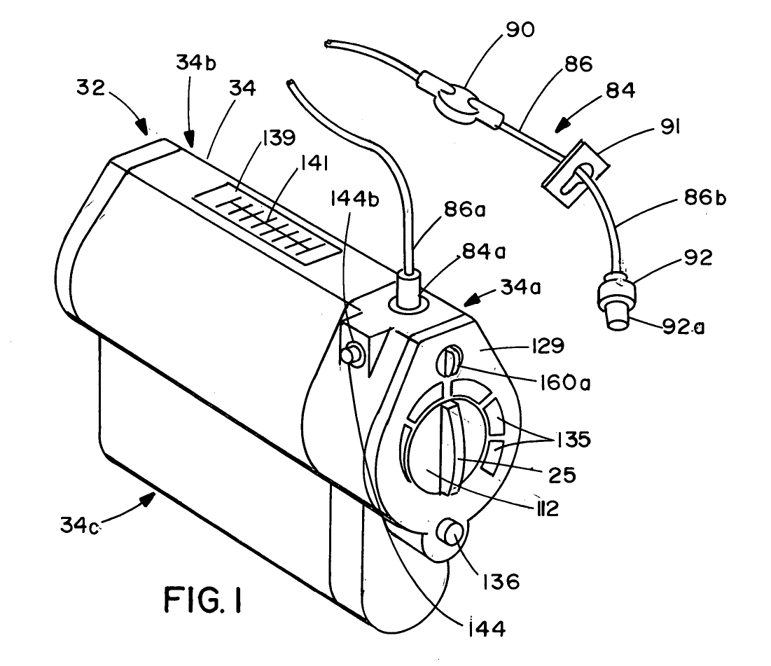

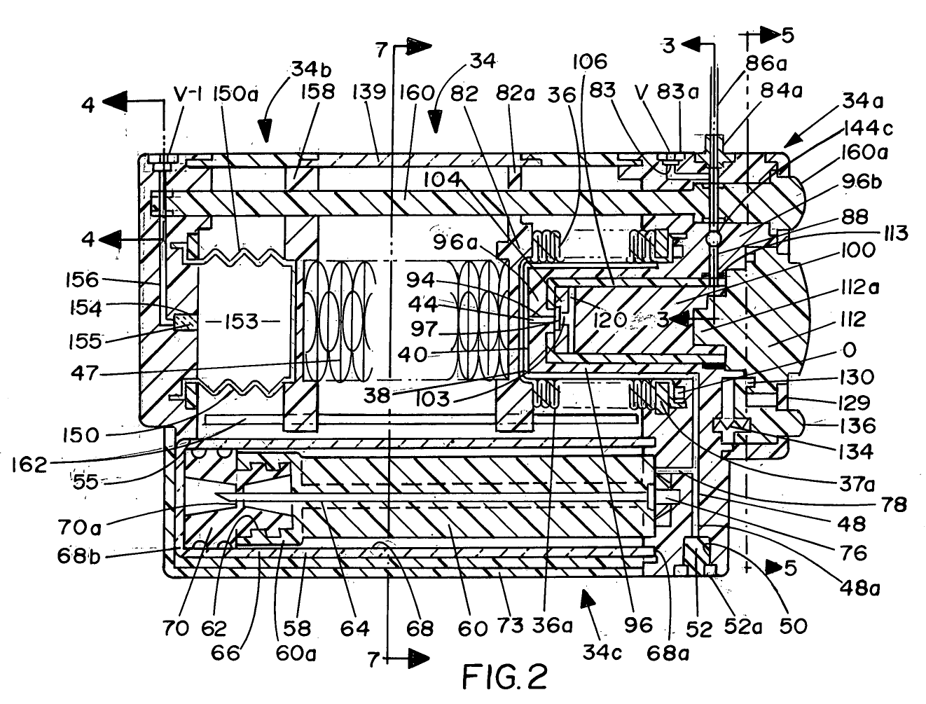

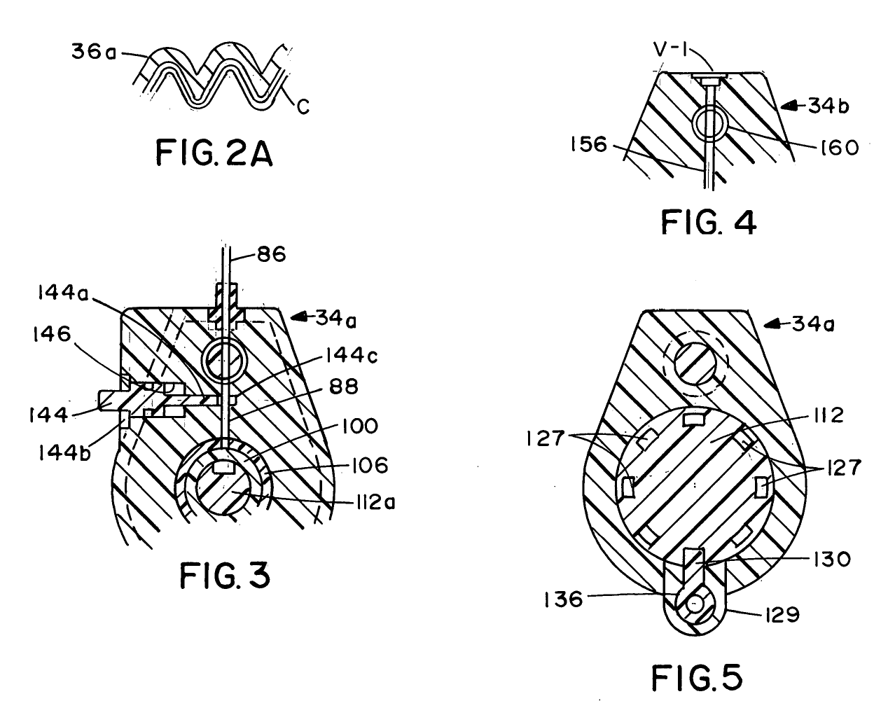

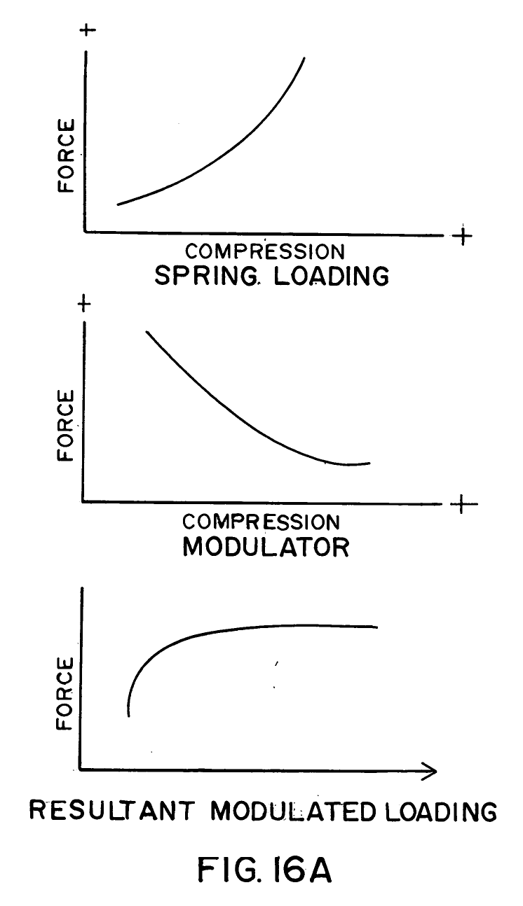

Referring to the drawings and particularly to FIGS. 1 and 2, one embodiment of the dispensing apparatus of the present invention is there illustrated and generally designated by the numeral 32. The apparatus here comprises a moldable plastic outer housing 34 having a first, second and third portions 34a, 34b and 34c respectively. Disposed within outer housing 34 is a first, expandable housing 36 having a fluid reservoir 38 (FIG. 15) provided with an inlet 40 for permitting fluid flow into the fluid reservoir and an outlet 44 for permitting fluid flow from the fluid reservoir. Expandable housing 36, which can be constructed from a metal or plastic material, can include a coating of the character presently to be described. Expandable housing 36 here comprises a bellows structure having an expandable and compressible, accordion-like, annular-shaped sidewall 36a, the configuration of which is best seen in FIGS. 15 and 16. The open end of the bellows is preferably sealably bonded to the device housing by an appropriate adhesive. Additionally, a sealing ring, such as ring 37a, prevents fluid leakage between the bellows and the device housing (FIG. 2).

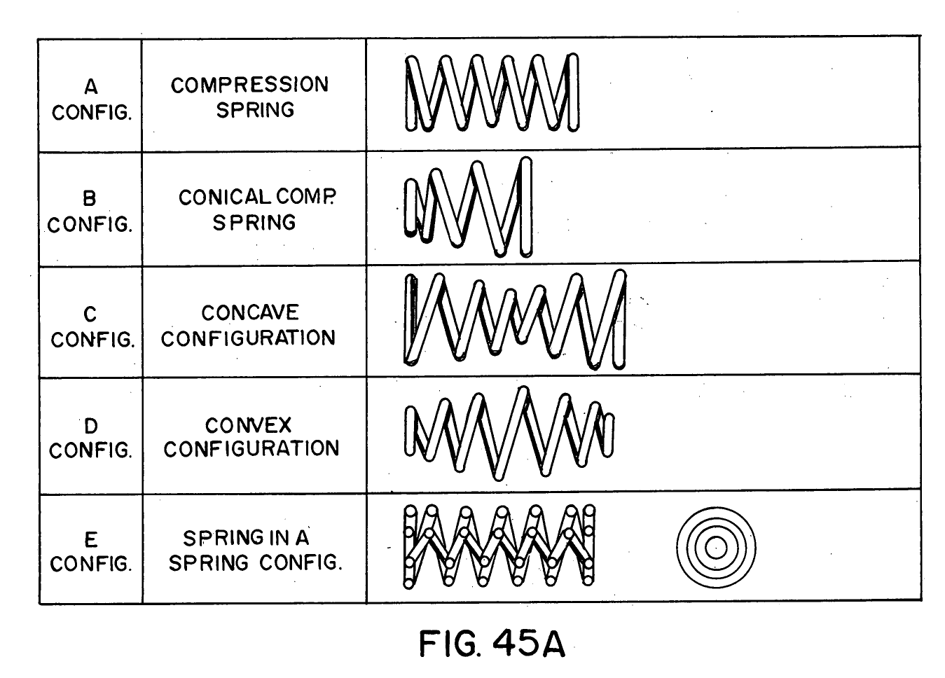

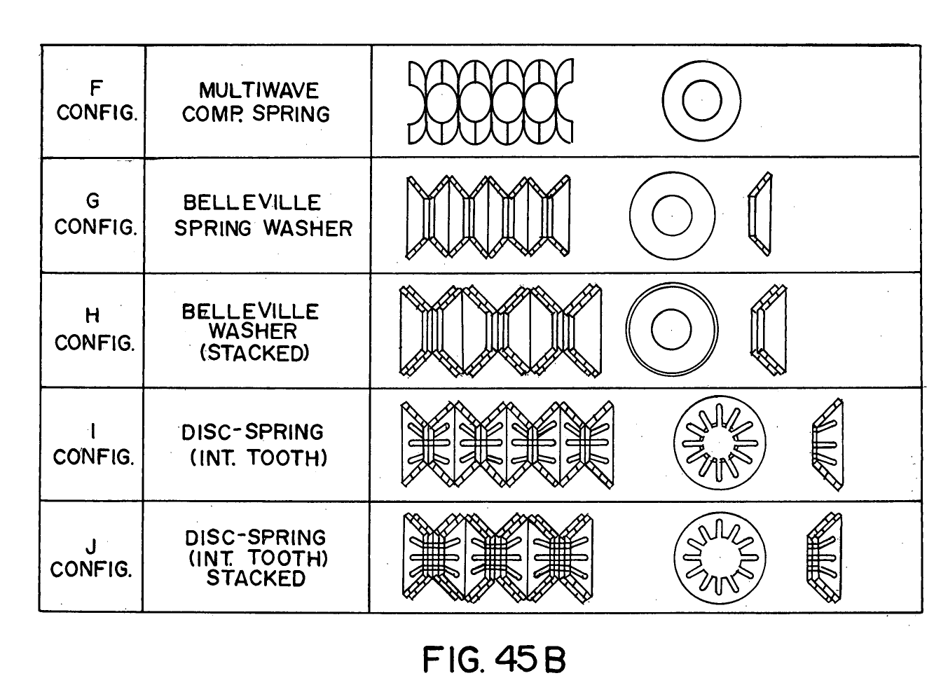

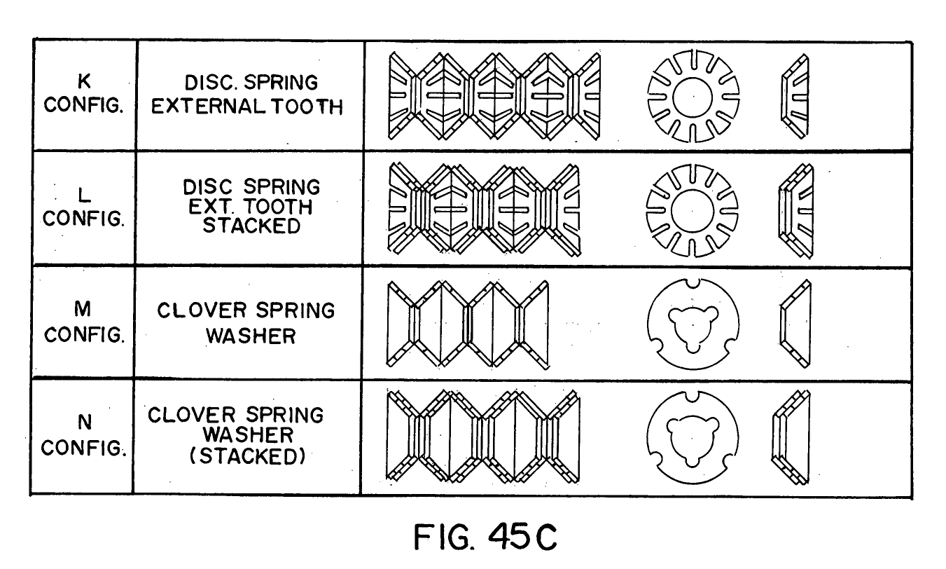

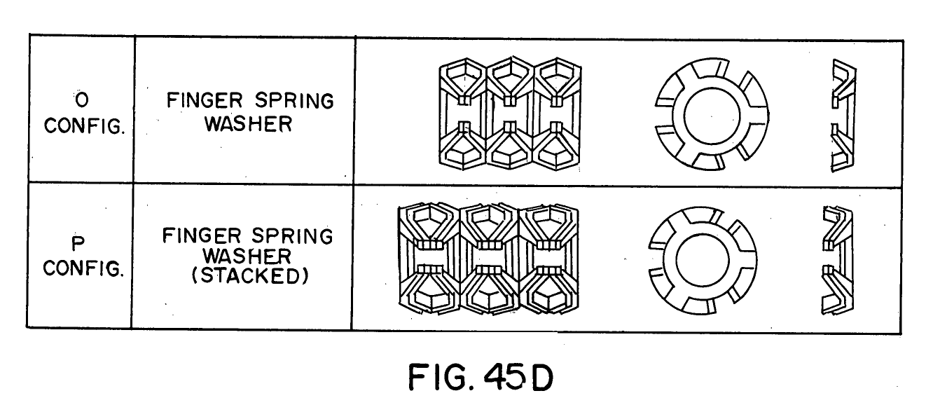

Disposed within second portion 34b of outer housing 34 is the novel, modulated stored energy means of the invention for acting upon inner expandable housing 36 in a manner to cause the fluid contained within fluid reservoir 38 to controllably flow outwardly of the housing. In the present form of the invention, this important stored energy means comprises a resiliently deformable, spring member 47 that is carried within the second portion 34b of the outer housing. In a manner presently to be described spring member 47 is controllably further compressed by fluid flowing into reservoir 38 and then is controllably expanded to cause fluid flow from the outer housing through the dispensing means of the invention. Stored energy member 47 can be constructed from a wide variety of materials including spring steel and plastic. In the preferred form of the invention, member 47 comprises a wave spring of the general type that is commercially available from various sources including the Smalley Company of Lake Zurich, Ill. However, as illustrated in FIGS. 45 and 46, and as will be discussed in greater detail hereinafter, several different types of springs can be used as the stored energy source of the invention.

Frequently, wave springs operate as loading devices. They can also take up play and compensate for dimensional variations within mechanical assemblies. A virtually unlimited range of forces can be produced whereby loads build either gradually or abruptly to reach a predetermined working height. This establishes a precise spring rate in which load is proportional to deflection. Typically, a wave spring will occupy an extremely small area for the amount of work it performs and will operate within a known deflection range. The use of this type of spring product is demanded, but not limited to tight axial and radial space restraints.

Forming an important aspect of the apparatus of the present invention is fill means carried by the third portion 34c of outer housing 34 for filling the reservoir 38 with the fluid to be dispensed. As best seen in FIG. 2, third portion 34c includes a fluid passageway 48 in communication with inlet 40 of fluid reservoir 38. Proximate its lower end 48a, fluid passageway 48 communicates with a cavity 50 formed within the third portion 34c of the housing. Disposed within cavity 50 is a pierceable elastomeric septum 52 that comprises a part of one form of the fill means and drug recovery of this latest form of the invention. Septum 52 is held in position by a suitably bonded retainer 52a and is pierceable by the needle of the syringe which contains the medicinal fluid to be dispensed and which can be used in a conventional manner to fill or partially fill reservoir 38 via passageway 48. The fill and recovery means of the invention can also comprise a slit septum and a mechanical check valve system of a type well known to those skilled in the art.

Third portion 34c of housing 34 also includes a chamber 55 for telescopically receiving a medicament containing closed-end shell fill vial 58. An elongated support 60, which is mounted within first chamber 55, includes a threaded end portion 62 and carries a longitudinally extending, elongated hollow needle or cannula 64 having a flow passageway that communicates with fluid passageway 48. Chamber 55, elongated support 60 and hollow needle 64 together comprise an alternate form of the fill means of the apparatus of the invention. The method of operation of this alternate form of fill means will presently be described.

Referring particularly to FIG. 2, the medicament containing plastic or glass shell fill vial 58 includes a body portion 66, having a fluid chamber 68 for containing the injectable fluid medicament. Chamber 68 is provided with a first open end 68a and second closed end 68b. First open end 68a is sealably closed by closure means here provided in the form of an externally threaded elastomeric plunger 70 which is telescopically movable within the vial from a first location where the plunger is disposed proximate first open end 68a to the second device-fill location shown in FIG. 2 where the plunger is disposed proximate second closed end 68b.

After opening of the slidable vial closure 73, which forms a part of the third portion 34c of housing 34 (FIG. 10), vial 58 can be inserted into chamber 55. As the fill vial is so introduced and the plunger 70 is threadably interconnected with end 60a of support 60, the sharp end of the elongated needle 64 will pierce the central wall 70a of the elastomeric plunger. Continuous pushing movement of the vial into chamber 55 will cause the structural support to move the elastomeric plunger inwardly of the vial chamber 68 in a direction toward the second or closed end 68b of the vial chamber. As the plunger is moved inwardly of the vial, the fluid contained within the vial chamber will be expelled there from into the hollow elongated needle 64. As best seen in FIG. 2, the fluid will then flow past elastomeric umbrella type check valve 76 and into a passageway 78 formed in third portion 34c of the apparatus housing. Umbrella type check valve 76 functions as a check valve to control fluid flow from the elongated hollow needle 64 toward fluid passageway 78. From passageway 78 the fluid will flow into passageway 48 and then into reservoir 38 of the bellows component 36 via ullage filling channel or inlet 40.

As the fluid flows into the bellows reservoir, the bellows will be expanded from the collapsed configuration shown in FIG. 2 into an expanded configuration (see FIG. 15). As the bellows member expands it will urge a telescopically movable volume indicator member or engagement coupling 82 that is carried within a second portion 34b of the housing and in engagement with the stored energy source, or spring member 47 causing it to compress. It is also to be understood that, if desired, the reservoir of the bellows component can be filled with an adjuvant drug or other appropriate fluid by alternate filling means of the character previously described which comprises a syringe having a needle adapted to pierce the pierceable septum 52 which is mounted within third portion 34c of the apparatus housing. As the reservoir 38 fills with fluid either from the fill vial or from the filling syringe, any gases trapped within the reservoir will be vented to atmosphere via vent means “V” mounted in portion 34b of the ullage member. This vent means here comprises a bonded gas vent 83 that can be constructed of a suitable hydrophobic porous material such as a porous plastic. Gas vent 83 is held in position within the housing by a bonded retainer ring 83a (FIG. 2).

Upon opening the fluid delivery path to the administration set 84 of the invention (FIG. 1) in a manner presently be described, the stored energy means, or member 47, will tend to return toward its starting configuration thereby controllably urging fluid flow outwardly of reservoir 38 via the flow control means of the invention the character of which will presently be described.

Administration set 84, which forms a part of the dispensing means of the invention for dispensing fluid to the patient, is connected to the first portion 34a of housing 34 by a connector 84a in the manner shown in FIG. 1 of the drawings. The proximal end 86a of administration line 86 of the administration set is in communication with an outlet fluid passageway 88 which is formed in housing portion 34a in the manner best seen in FIG. 2. Disposed between the proximal end 86a and the distal end 86b of the administration line is a conventional gas vent and particulate filter 90 and a conventional clamp 91. Provided at the distal end 86b is a luer connector 92 and a cap 92a of conventional construction (FIG. 1).

As previously discussed, a number of liquid injectable beneficial agents can be contained within shell vial 58 and can be controllably dispensed to the patient including, by way of example, medicaments of various types, drugs, pharmaceuticals, hormones, antibodies, biologically active materials, elements, chemical compounds, or any other suitable material useful in diagnostic cure, midigation, treatment or preventing of diseases or the maintenance of the good health of the patient.

As the fluid contained within the bellows reservoir 38 is urged outwardly thereof by the stored energy means, the fluid will flow into a fluid passageway 94 formed in the first portion 96a of an ullage member 96. Ullage member 96 forms a part of the first portion 34a of the housing 34 and includes a first portion 96a, which is housed within bellows 36, and within which the bellows slidably cooperates (FIG. 2). First portion 96a functions as a ullage member to ensure that substantially all of the residual fluid contained within the fluid reservoir is appropriately dispensed. The fluid will next flow under pressure through a filter means shown here as a filter 97 that is peripherally bonded with a cavity provided in the flow control member 100 of the flow control assembly 104. Filter 97, which functions to filter particulate matter from the fluid flowing outwardly from reservoir 38, is of a character well known to those skilled in the art and can be constructed from various readily available materials such as polysolfone and polypropylene wafers having a desired porosity. After flowing through filter 97, the fluid will flow, via a stub passageway 103 (FIG. 2) into the novel flow control means of the invention that is disposed interiorly of housing 34. This important flow control means functions to precisely control the rate of fluid flow outwardly from reservoir 38 and toward the patient.

If the internal materials interface of the bellows structure and other fluid channels or surfaces are not sufficiently compatible with the planned beneficial agent to be delivered, either in terms of its biocompatibility or drug up-take characteristics, application of a surface modification process is appropriate. This surface modification methodology to provide a barrier coating “C” as shown in FIG. 2A, may take one of several forms including single or multiple layer coatings. One process that is extremely clean, fast and efficient is plasma processing. In particular this technique allows for any of the following: plasma activation, plasma induced grafting and plasma polymerization of molecular entities on the internal drug surface of the bellows. For cases where an inert hydrophobic interface is desired, plasmas using fluorine-containing molecules may be employed. That is, the bellows surface as well as other surfaces or fluid passageways that may be contacted by the beneficial agent may be cleaned with an inert gas plasma, and subsequently, a fluorine-containing plasma may be used to graft these molecules to the surface. Alternatively, if a hydrophilic surface is desired (e.g. for drug solutions that are highly corrosive or in oil-based solvents) an initial plasma cleaning may be done, followed by a plasma polymerization using hydrophilic monomers.

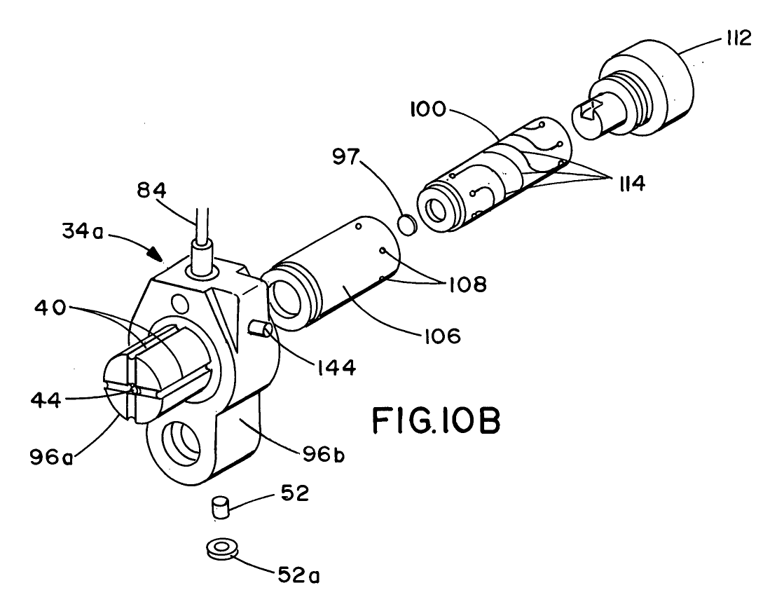

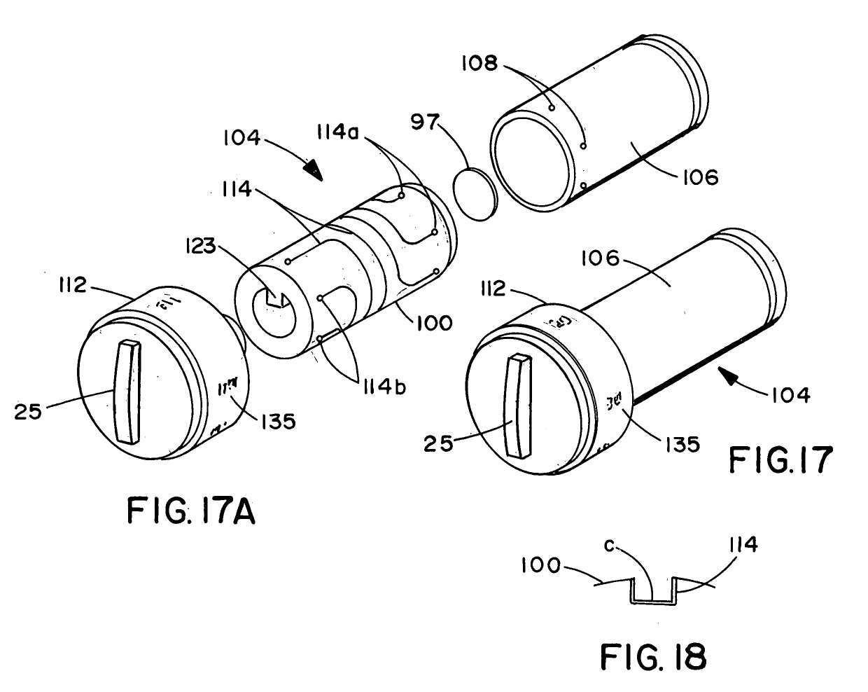

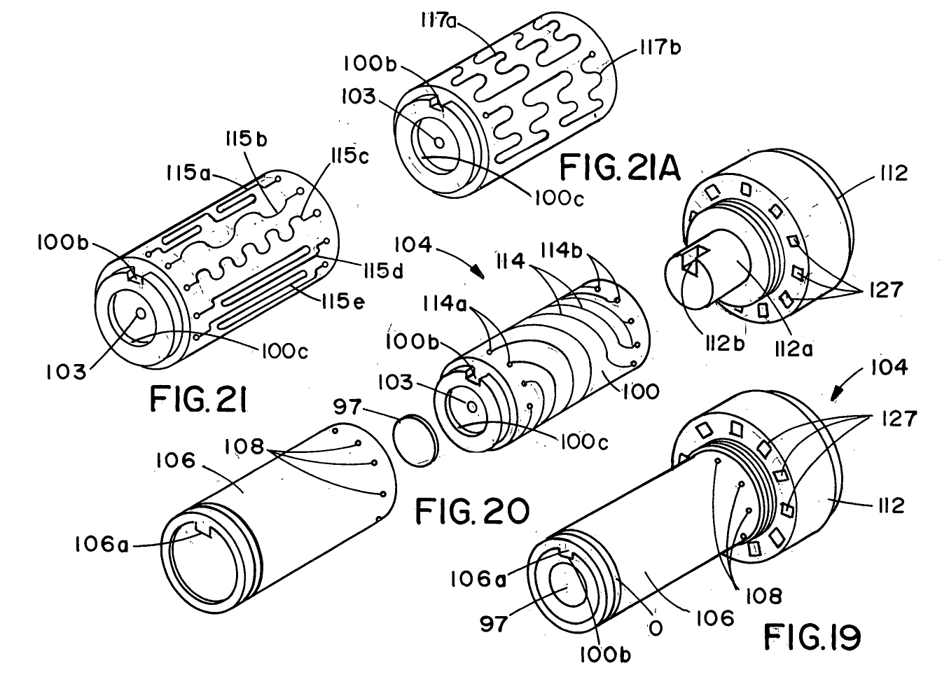

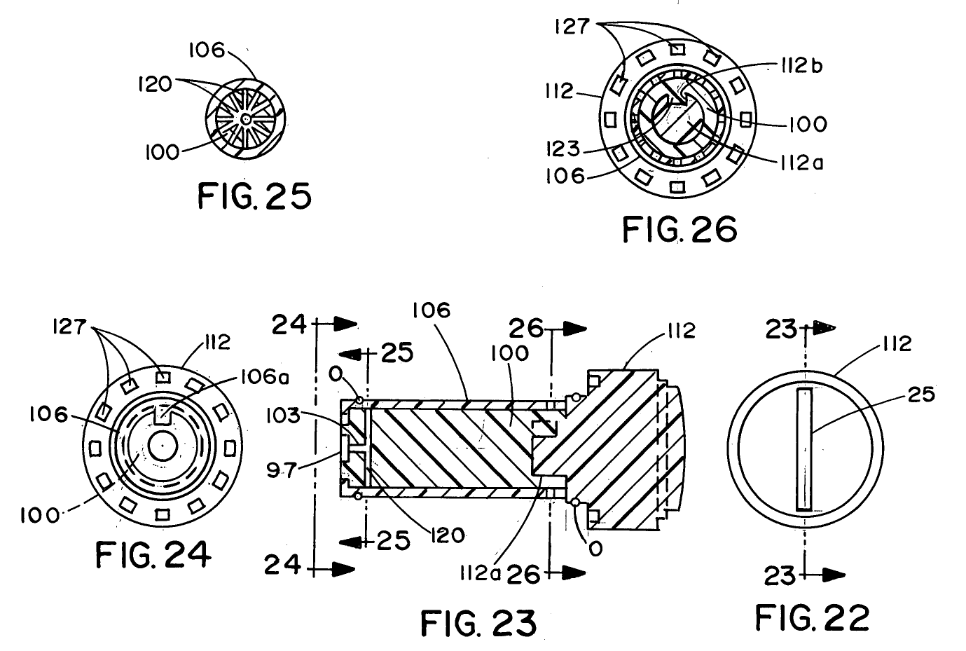

Referring to FIGS. 17 through 26, it can be seen that flow control assembly 104 comprises an outer casing 106 having a plurality of circumferentially spaced apart fluid outlets 108, a flow control member 100, which is telescopically receivable within casing 106 and a selector knob 112 that is interconnected with control member 100 in the manner best seen in FIG. 23. As illustrated in FIGS. 17A and 20, flow control member 100 is uniquely provided with a plurality of elongated, micro-fluidic flow control channels 114, each having an inlet 114a and an outlet 114b. The flow channels may be of different sizes, lengths, widths, depths and configurations as shown by FIG. 21, which depicts an alternate form of the flow control member having flow channels 115a, 115b, 115c, 115d, and 115e. The flow channels identified by the numerals 117a amd 117b in FIG. 21A, which illustrates yet another form of flow control member of the invention, can be of still another configuration. Here the flow channels define circuitous flow paths in a plurality of individually, spaced-apart flow segments. Further, the flow control channels may be rectangular in cross-section as illustrated in FIG. 18, or alternatively, they can be semicircular in cross-section, U-shaped in cross-section, or they may have any other cross-sectional configuration that may be appropriate to achieve the desired fluid flow characteristics. The flow control channels may also be coated, if appropriate, with a coating “C” or alternate surface treatment (see FIG. 11) of the character previously described herein. When the flow control member is properly positioned and bonded within outer casing 106, the inner surface of the outer casing wall cooperates with channels 114 (FIG. 20) to form a plurality of generally spiral shaped fluid flow passageways of different overall lengths and flow capacities. When the flow control member is positioned within the outer casing, a notch 100b formed in member 100 receives a tongue 106a provided on casing 106 so precisely align the outlets 114b of the flow channels 114 with fluid outlets 108 formed in casing 106. It is to be understood, the suitable O-rings, generally designated as “O” are used to sealably interconnect the completed assembly (see FIG. 19) to outer housing 96.

Selector knob 112, which comprises a part of the selector means of the invention, is rotatably sealably connected to second portion 96b of ullage defining member 96 by means of an elastomeric band 113 and, in a manner presently to be described, functions to rotate the assembly made up of outer casing 106 and flow control member 100. In this way, a selected outlet 108 in casing 106 can be selectively aligned with the flow passageway 88 provided in the ullage-defining member (see FIG. 2).

As previously discussed herein, as the fluid contained within the bellows reservoir 38 is urged outwardly thereof by the stored energy means, the fluid will flow into a fluid passageway 94 formed in the first portion 96a of an ullage member 96. The fluid will next flow under pressure through filter 97 that is bonded within cavity 100c (FIG. 20) provided in the flow control member 100 of the flow control assembly 104.

After flowing through filter 97, the fluid will flow, via stub passageway 103 into the distribution means of the invention for distributing fluid from the fluid reservoir to each of the plurality of spiral passageways 114 (FIG. 20). This distribution means here comprises several radially outwardly extending flow passageways 120 formed in flow control member 100 (FIG. 25). The filtered fluid will fill passageways 120 and then will flow into the plurality of spiral passageways 114 via ports 114a formed in member 100 and then outlets 114b, which communicate with passageways 114 (see FIG. 20). The fluid contained within spiral passageways 114 can flow outwardly of the device only when one of the fluid outlets 108 formed in casing 106 is aligned with reservoir outlet passageway 88 (FIGS. 2 and 19). A single apertured elastomeric sealing band 113 provides for rotating sealing between ullage 96 and housing 106. As indicated in FIG. 2, the aperture provided in band 113 aligns with fluid passageway 88.

The flow control channels 114 can be made by several techniques including (micro) injection molding, injection-compression molding, hot-embossing and casting. The techniques used to make these imbedded fluid channels are now common-place in the field of microfluidics, which gave rise to the lab-on-a-chip, bio-MEMS and micro-total analysis systems (m-TAS) industries. Additionally, depending on the size of the fluid channels required for a given flow rate, more conventional micro injection molding techniques can be used.

The first step in making the channels using an injection molding or embossing process is a lithographic step, which allows a precise pattern of channels to be printed on a “master” with lateral structure sizes down to 0.05 mm. subsequently, electroforming is performed to produce the negative metal form, or mold insert. Alternatively for larger chanel systems, precision milling can be used to make the mold insert directly. Typical materials for the mold insert or embossing tool are nickel, nickel alloys, steel and brass. Once the mold insert of embossing tool is fabricated, the polymer of choice may be injection molded or embossed to yield the desired part with imprinted channels.

Alternatively, channels can also be made by one of a variety of casting processes. In general, a liquid plastic resin (e.g. a photopolymer) can be applied to the surface of a metal master (made by the techniques described above) and then cured via thermal of UV means. After hardening, the material is then “released” from the mold to yield the desired part. Additionally, there are similar techniques available that utilize CAD data (of the desired channel configuration) and direct laser curing of a liquid monomer to yield a polymerized and solidified part with imbedded channels. This process is available by contract, for example, for MicroTEC MbH of Duisburg, Germany.

A number of materials can be used to fabricate flow control member 86. While medical grade polymers are the most appropriate materials, other materials can be used including: Thermoplastics (embossing & injection molding); Duroplastics (injection molding); Elastomers (injection compression molding and soft lithography); Polyurethanes (castings); and Acrylics and Epoxies. U.S. Pat. No. 6,176,962 and WO 99/5694 disclose various techniques for making micro-fluidic flow channels

Selection of the passageway 114 from which the fluid is to be dispensed is accomplished by rotation of the selector knob 112 which, as best seen in FIGS. 20 and 23 includes a reduced diameter portion 112a having a slot 112b formed therein. As illustrated in FIGS. 17A and 26, slot 112b is adapted to receive a spline 123 (FIG. 17A) formed anteriorly of member 100. With this construction, rotation of selector member 112 by gripping a transversally extending finger grip ping member 25 will impart part rotation to member 112. As seen in FIG. 20, inwardly extending spline segment 106a is received within slot 100b formed in the rearward periphery of member 100. Accordingly, rotation of member 112 will also impart concomitant rotation to casing member 106.

As illustrated in FIGS. 20 and 26, selector knob 112 is provided with a plurality of circumferentially spaced apart indexing cavities 127 that closely receive an indexing finger 130 which forms a part of the indexing means of the invention, which means comprises a locking shaft cover 129 that is connected to third portion 34c of the apparatus housing (see FIGS. 2 and 5). Indexing finger 130 is continuously urged into engagement with a selected one of the indexing cavities 127 by a coil spring 134 that also forms a part of the indexing means of the invention. Coil spring 134 can be compressed by an inward force exerted on an indexing shaft 136 that is mounted in locking shaft cover 129 and is movable from the extended position shown in FIG. 2 to an inward, finger release position wherein spring 134 is compressed and finger 130 is retracted from a selected indexing cavity 127. With finger 130 in its retracted position it is apparent that control knob 112 can be freely rotated to a position wherein gripping member 25 can be aligned with selected flow rate indicia 135 formed on the front bezel 129 of the apparatus housing (FIG. 1).

When the selector knob is in the desired position and pressure is released on indexing shaft 136, spring 134 will urge finger 130 of the indexing means of the invention into locking engagement with one of the indexing cavities 127 thereby placing a selected one of the spiral shaped flow control channels 114 in communication with the fluid reservoir 38 via passageways 44, 103 and 120. As the fluid flows outwardly of the apparatus due to the urging of the stored energy means or spring member 47, the bellows structure 36 will be collapsed and at the same time coupling member 82 will travel inwardly of housing portion 34b. Member 82, which forms a part of the volume indicator means of the invention, includes a radially outwardly extending indicating finger 82a that is visible through a volume indicator window 139 that is provided in a second portion 34b of the apparatus housing and also comprises a part of the volume indicator means of the invention (FIGS. 1 and 2). Indicia 141, which are provided on indicator window 139, function to readily indicate to the caregiver the amount of fluid remaining within fluid reservoir 38. Referring to FIG. 3, disabling means, shown here as a disabling shaft 144 that is telescopically movable within a passageway 146 formed within housing portion 34a functions to disable the device. More particularly, shaft 144 has a distal end 144a, which, upon insertion of the shaft, will block fluid flow through passageway 88. A bonded retainer 144b normally holds shaft 144 in the retracted position.

Considering next the important modulating means of the invention for modulating the force exerted upon inner expandable housing 36 by the stored energy means, or spring 47. In the present form of the invention this modulating means comprises a second expandable housing 150 that is carried by outer housing 34 and is operably associated with first expandable housing 36. Second expandable housing comprises a bellows structure having an accordion like sidewall 150a that defines a fluid chamber 153 for containing a fluid such as air. Second expandable housing 150, which has an outlet 155 for permitting the flow of air there through, is movable from the substantially expanded configuration shown in FIG. 2 to the substantially collapsed configuration shown in FIG. 16, by a force exerted thereon by spring member 47. The modulating means of the present form of the invention further includes impedance means, here provided as an impedance porous frit 154, that is disposed within fluid outlet 155, for controllably impeding the flow of the fluid contained within fluid chamber 153 outwardly thereof to atmosphere via a flow passageway 156 formed in second housing portion 34b and a vent V-1.

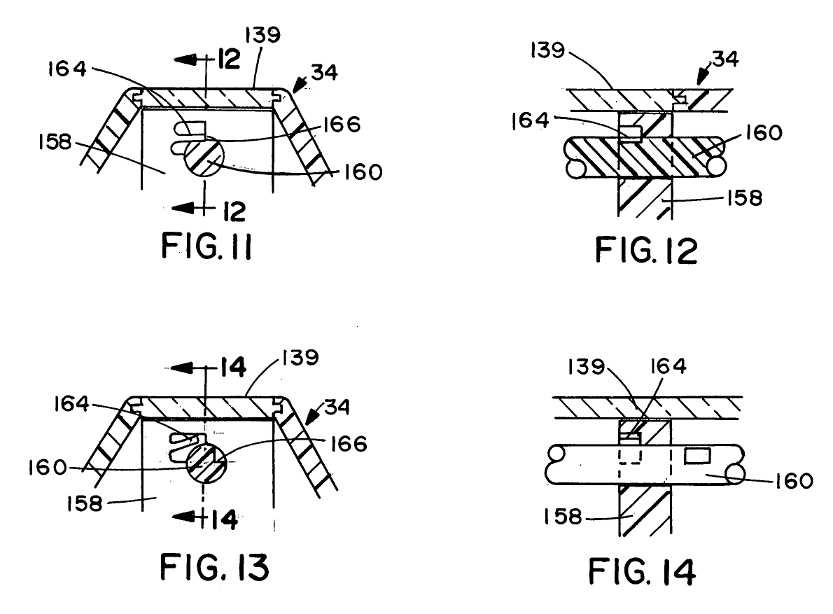

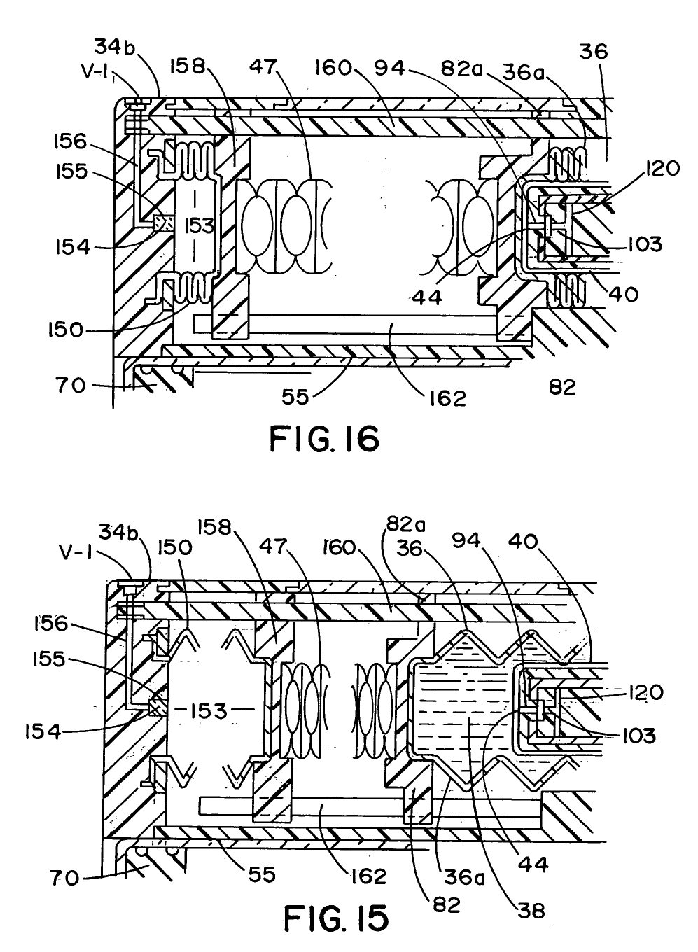

Disposed between spring 47 and second bellows housing 150 is an air collar 158 that is slidably movable within housing 34 along upper and lower, longitudinally extending shafts 160 and 162 (see FIGS. 15 and 16). During the medicament delivery step, spring 47 acts upon indicator member 82, which, in turn acts upon first bellows assembly 36 tending to collapse it and to cause the medicinal fluid contained within reservoir 38 to be forced outwardly thereof via reservoir outlet 44. At the same time, spring 47 acts upon air collar 158 which, in turn, acts upon second to bellows 150 tending to collapse it. However, before air collar 158 can slidably move along control shafts 160 and 162, the air collar must be released from its normally locked position shown in FIGS. 11 and 12 of the drawings. As indicated in FIGS. 11 and 12, sliding movement of air collar 158 is normally prevented by locking means shown here as a stop tab 164 that engages a shoulder 166 formed on control rod 160. At the commencement of the medicament delivery step, control rod 160 is rotated by gripping the finger grip portion 160a thereof. As indicated in FIGS. 13 and 14, when the control shaft 160 is controllably rotated, the stop tab 164 to ride up on the shaft and out of locking engagement with shoulder 166 allowing the air collar to move rearwardly of the control shaft in the manner illustrated in FIG. 14.

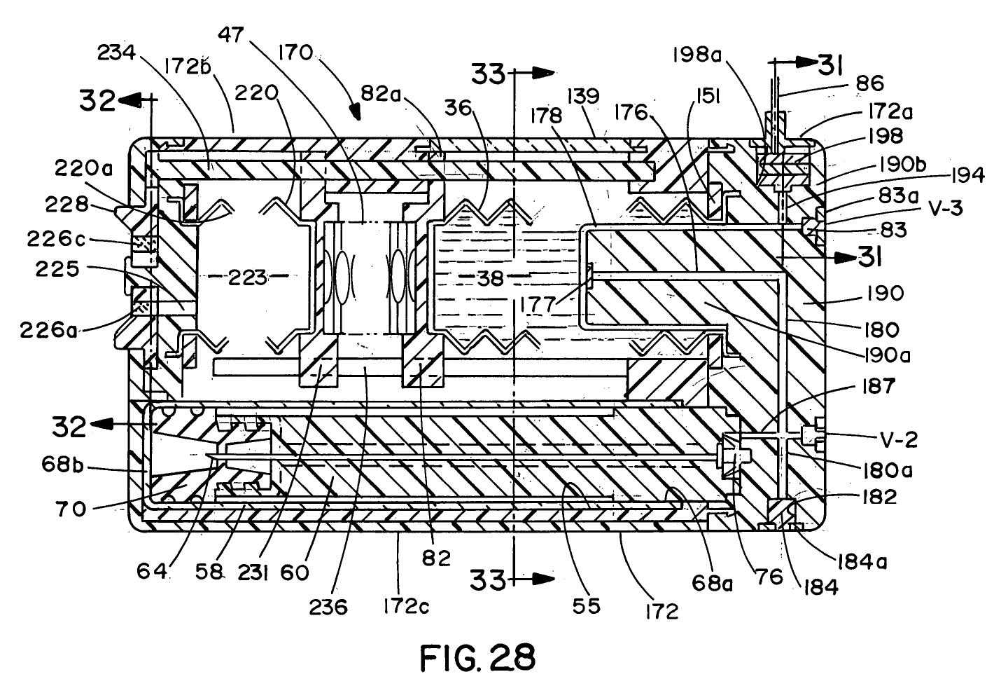

Rearward movement of the air collar due to the urging of spring 47, in the manner illustrated in FIG. 15, will cause the air within chamber 153 of the second bellows assembly 150 to controllably flow through porous frit 154, which is appropriately tuned to the particular spring constant, and outwardly to atmosphere via the vent V-1. As the air collar moves rearwardly of the housing, it is apparent that the force being exerted on first bellows 36 by spring 47 will be modulated. As shown in FIG. 16A of the drawings, this modulation of the force exerted by spring 47 on second bellows 36 uniquely results in a more linear flow of medicinal fluid outwardly of the device as depicted in the lower-most graph of FIG. 16A. More particularly, as shown in the upper-most graph of FIG. 16A, the greater the compression on the spring, the greater will be the force generated by the spring. Accordingly, at the beginning of the fluid delivery cycle, when the spring is highly compressed, the force generated by the spring will be greater than the force generated as the spring relaxes and approaches the end of the fluid delivery cycle. This spring unloading, unless compensated for, will result in a greater fluid flow at the beginning of the fluid delivery cycle and a lesser fluid flow toward the end of the delivery cycle. Second bellows assembly 150 of the modulating means functions to compensate for this undesirable condition. More particularly, as the second bellows, is compressed by the spring in the manner shown in FIGS. 15 and 16, the second bellows assembly 150 functions to counter act, or modulate the greater force generated by the spring during the early portion of the flow delivery cycle. This novel modulating action as depicted in the lower-most graph of FIG. 16A, results in the vastly improved constant flat linear flow of the medicinal fluid outwardly of the apparatus. When all of the medicinal fluid has been delivered from the fluid reservoir 38, spring 47 will have expanded into the configuration shown in FIG. 16 and both of the first and second bellows assemblies 36 and 150 will have been fully collapsed. As shown in FIG. 28, a suitable seal ring 151 is provided to prevent leakage between the bellows and housing portion 194.

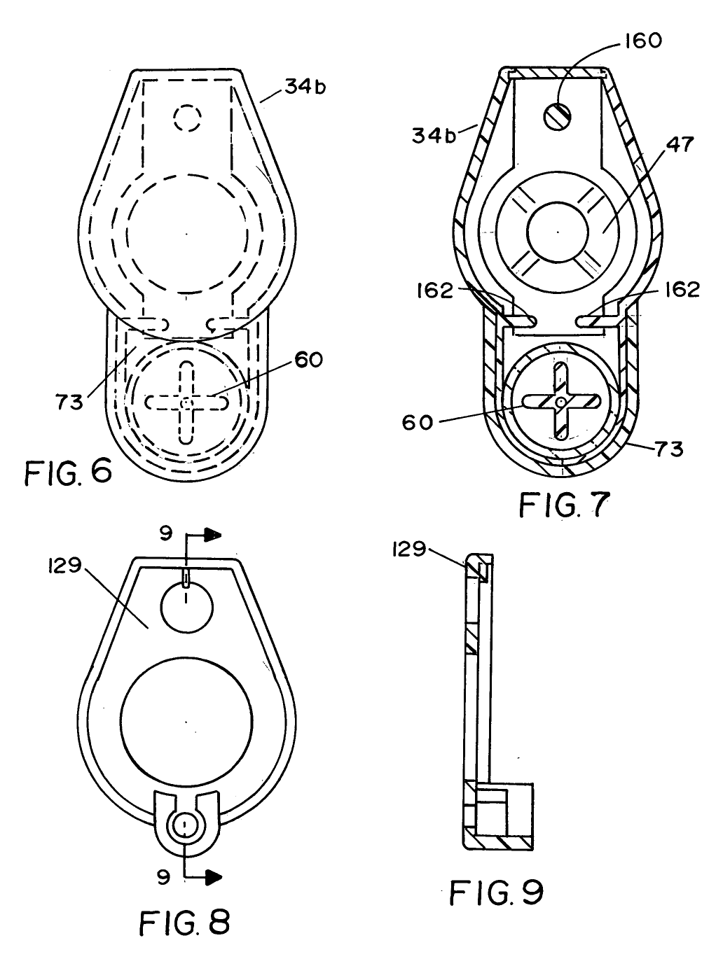

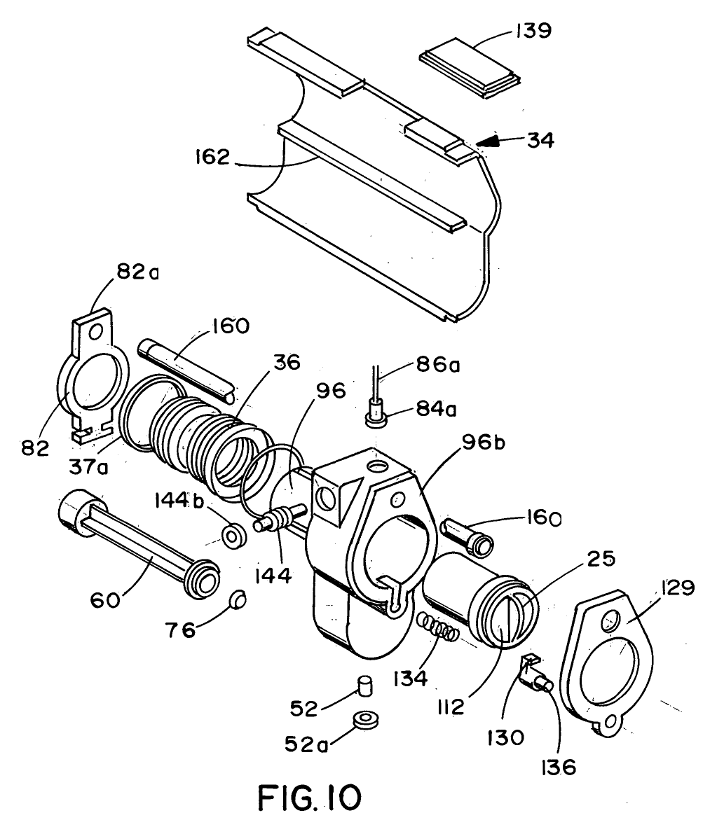

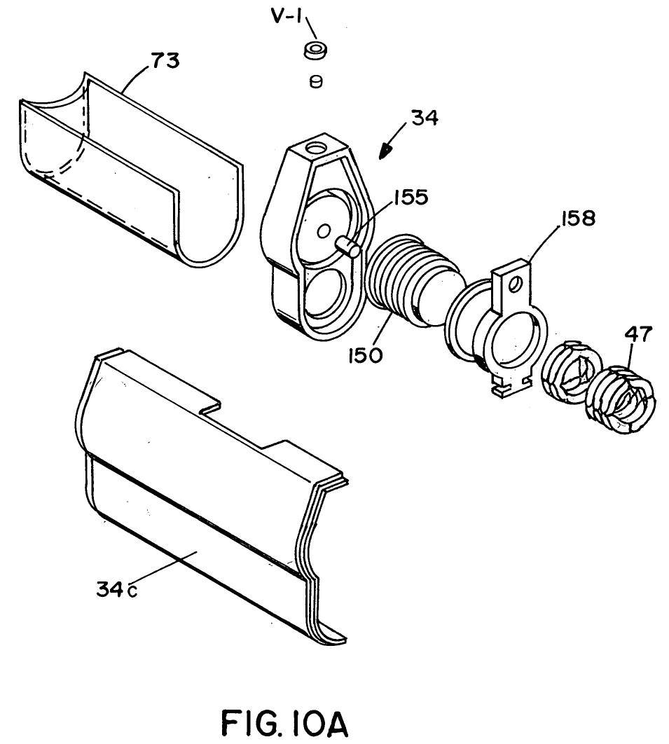

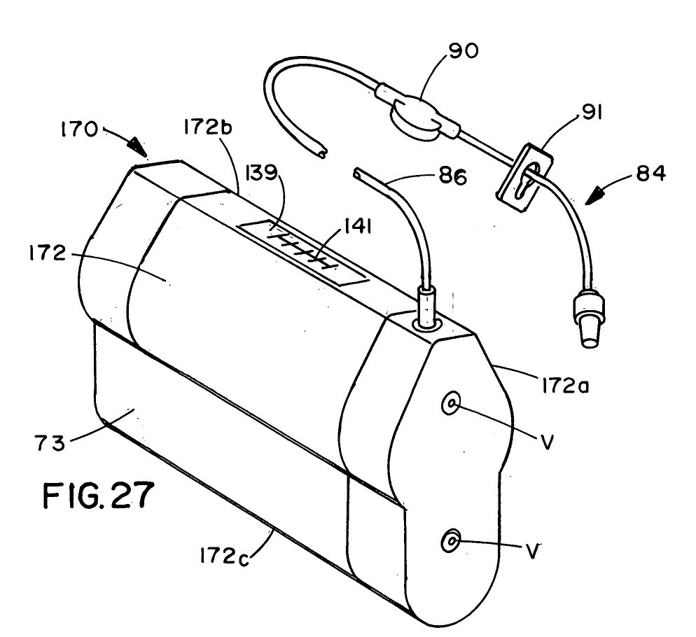

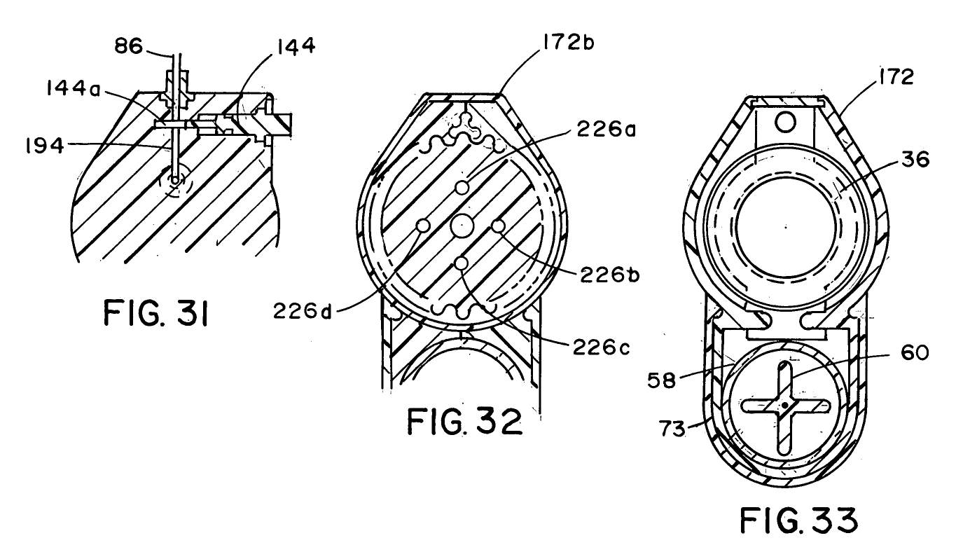

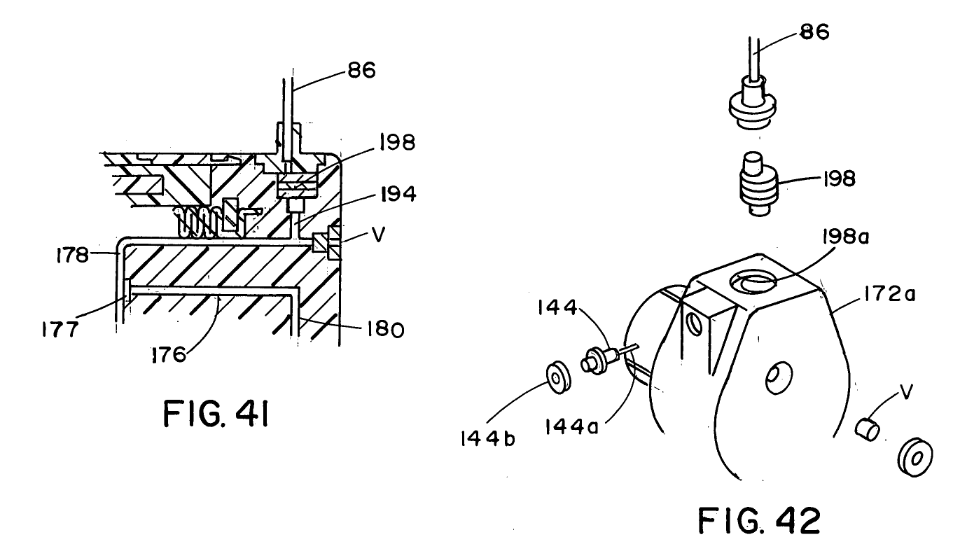

Referring now to FIGS. 27 through 44, another embodiment of the dispensing apparatus of the present invention is there illustrated and generally designated by the numeral 170. This alternate form of the apparatus of the invention is similar in many respects to that shown in FIGS. 1 through 26 and like numerals are used in FIGS. 27 through 44 to identify like components. The primary differences between this latest form of the invention and that shown in FIGS. 1 through 26 concern the provision of a differently configured flow rate control means for controlling the rate of fluid flow from the apparatus and the provision of a differently designed control mechanism for controlling the flow of fluid outwardly of the second bellows assembly of the apparatus. More particularly, this alternate form of control mechanism is operable from the rear of the apparatus rather than from the front. Additionally, as will be better understood from the discussion, which follows, this latest embodiment of the invention includes a plurality of flow control, porous frits that are strategically positioned relative to the second bellows to control fluid flow from the second bellows.

As best seen by referring to FIGS. 27 and 28, the apparatus of this latest form of the invention comprises an outer housing 172 having a first, second and third portions 172a, 172b and 172c respectively. Disposed within outer housing 172 is a first, expandable housing 36, which is a similar construction to that previously described and includes a collapsible bellows like structure that defines a fluid reservoir 38. As before, reservoir 38 is provided with an inlet passageway 176 for permitting fluid flow into the fluid reservoir and an outlet 178 for permitting fluid flow from the fluid reservoir.

Disposed within second portion 172b of outer housing 172 is the modulated stored energy means of the invention for acting upon first expandable housing 36 in a manner to cause the fluid contained within fluid reservoir 38 to controllably flow outwardly of the housing. In this latest form of the invention, this important stored energy means is generally similar to that previously described and comprises a compressively deformable, spring member 47 that is carried within the second portion 172b of the outer housing. As before, spring member 47 is first compressed by fluid flowing into reservoir 38 and then is controllably expanded to cause fluid flow from the outer housing through the dispensing means of the invention.

As in the earlier described embodiment of the invention, fill means are carried by the third portion 172c of outer housing 172 for filling the reservoir 38 with the fluid to be dispensed. In this regard, third portion 172c includes a fluid passageway 180 in communication with inlet passageway 176 of fluid reservoir 38. Proximate its lower end 180a, fluid passageway 180 communicates with a cavity 182 formed within the third portion 172c of the housing. Disposed within cavity 182 is an elastomeric, pierceable septum 184 that comprises a part of one form of the fill means of this latest form of the invention. Septum 184 is held in position by a retainer 184a and is pierceable by the needle of the syringe which contains the medicinal fluid to be dispensed and which can be used in a conventional manner to fill or partially fill reservoir 38 via passageway 180.

Third portion 172c of housing 172 also includes a chamber 185 for telescopically receiving a medicament containing fill vial 58, which is identical in construction and operation to that previously described, as is the elongated support 60, which is mounted within first chamber 55. Chamber 55, elongated support 60 and hollow needle 64 together comprise an alternate form of the fill means of the apparatus of this latest form of the invention.

During the reservoir filling step in the manner previously described, as the elastomeric plunger is moved inwardly of the vial, the fluid contained within the vial chamber will be expelled there from into the hollow elongated needle 64. As best seen in FIG. 28, the fluid will then flow past umbrella type check valve 76 and into a passageway 187 formed in third portion 172c of the apparatus housing. Umbrella type check valve 76 functions to control fluid flow from the elongated hollow needle 64 toward fluid passageway 187. From passageway 187 the fluid will flow into passageway 180 and then into reservoir 38 of the bellows component 36 via inlet passageway 176 and a suitable filter 177. Any gas is contained within the fill vial can be vented to atmosphere and via a vent “V-3”.

As the fluid flows into the bellows reservoir, the bellows will be expanded from a collapsed configuration into an expanded configuration shown in FIG. 28. As the bellows member expands it will urge a telescopically movable volume indicator member 82 that is carried within a second portion 172b of the housing into engagement with the stored energy source, or spring member 47 causing it to compress. It is also to be understood that, if desired, the reservoir of the bellows component can also be filled by alternate filling means of the character previously described which comprises a syringe having a needle adapted to pierce the pierceable septum 184 which is mounted within third portion 172c of the apparatus housing. As the reservoir 38 fills with fluid either from the fill vial or from the filling syringe, any gases trapped within the reservoir will be vented to atmosphere via vent means “V-3” that is mounted in portion 190b of an ullage member 190. This vent means here comprises a gas vent 83 that can be constructed of a suitable hydrophobic porous material such as a porous plastic. Gas vent 83 is held in position within the housing by a retainer ring 83a (FIG. 28).

Upon opening the fluid delivery path to the administration set 84 of the invention (FIG. 27), which is identical to that previously described, the stored energy means, or member 47, will tend to return to its starting configuration thereby controllably urging fluid flow outwardly of reservoir 38 via the flow control means of the invention the character of which will presently be described.

As the fluid contained within the bellows reservoir 38 is urged outwardly thereof by the stored energy means, the fluid will flow into an outlet passageway 192 and then into a stub passageway 194 formed in portion 190b of the ullage member 190. Ullage member 190 includes, in addition to portion 190b, a second portion 190a that is housed within bellows 36 (FIG. 28). After flowing into stub passageway 194, the medicinal fluid will flow into the novel flow control means of the invention that is disposed within ullage portion 190b. This important flow rate control means functions to precisely control the rate of fluid flow outwardly from reservoir 38 and toward the patient.

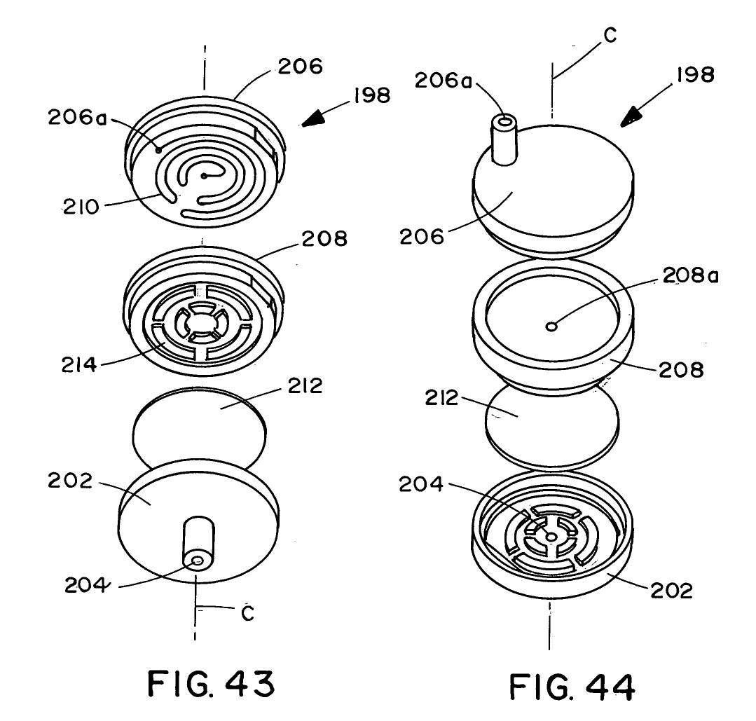

Referring to FIGS. 28, 41, 42, 43 and 44, it can be seen that the flow rate control means here comprises a rate control assembly 198 that is housed within a cavity 198a formed in ullage portion 190b. As best seen in FIGS. 43 and 44, this novel rate control assembly comprises an inlet manifold 202 having an inlet port 204 that is in communication with an outlet manifold 206 that is interconnected with intake manifold 202 by means of a separator plate 208. As indicated in FIGS. 28 and 44, outlet manifold 206 as an outlet port 206a that is in communication with administration line 86 of the administration set 84. As shown in FIG. 43, outlet manifold 206 is provided with an elongated micro channel 210 that is in communication both with inlet port 204 and with outlet port 206a of the outlet manifold. It is to be understood that, while micro fluidic channel is here shown in a spiral configuration, it can be provided in a number of different types of configurations and, if desired, can be appropriately coated. Disposed intermediate inlet manifold 202 and the generally circular shaped separator plate 208 is filter means here provided as a filter member 212 that functions to filter fluid flowing toward outlet port 206a of the outlet manifold. Generally disk shaped filter member 212 can be formed from various porous materials, including porous metals and porous ceramics.

As best seen in FIG. 43, separator plate 208 is provided with standoff ribs 214 for supporting filter member 212. The assemblage made up of inlet manifold 202, outlet manifold 206, separator plate 208 and filter 212 is encapsulated within housing cavity 198a in the manner shown in FIG. 28.

As indicated in FIG. 43, the flow rate control means, or assemblage 198, has an axial centerline “C” with which the inlet port 204 of the inlet manifold 202 is coaxial aligned. However, the outlet port 206a of the outlet manifold 206 is radially spaced from the axial centerline. With this construction, fluid will flow from reservoir 38 into inlet port 204, through filter member 212, through a central opening 208a formed in separator plate 208 and thence into micro channel 210. By controlling the length and depth of the micro channel 210, the rate of fluid flow flowing outwardly of outlet 206a can be precisely controlled. In this regard, the micro channel can take several forms and is not limited to the configuration shown in FIG. 43 of the drawings.

Turning once again to FIG. 27, the dispensing means for dispensing fluid to the patient comprises the previously identified administration set 84 that is connected to the first portion 172a of housing 172 in the manner shown in the drawings. As previously discussed, a number of beneficial agents can be controllably dispensed to the patient including, by way of example, medicaments of various types, drugs, pharmaceuticals, hormones, antibodies, biologically active materials, elements, chemical compounds, or any other suitable material useful in diagnostic cure, medication, treatment or preventing of diseases or the maintenance of the good health of the patient.

During the fluid delivery step, as the fluid flows outwardly of the apparatus due to the urging of the stored energy means or spring member 47, the bellows structure 36 will be collapsed and at the same time member 82 will travel inwardly of housing portion 172b. Member 82, which forms a part of the volume indicator means of the invention, includes a radially outwardly extending indicating finger 82a that is visible through a volume indicator window 139 that is provided in a second portion 172b of the apparatus housing and also comprises a part of the volume indicator means of the invention (FIGS. 27 and 28). Indicia 141, which are provided on indicator window 139, function to readily indicate to the caregiver the amount of fluid remaining within fluid reservoir 38.

Referring to FIG. 42, disabling means of the same construction and operation as that previously discussed in connection with the first embodiment of the invention are provided to disable the device. More particularly, shaft 144 has a distal end 144a, which, upon insertion of the shaft, will block fluid flow through passageway 194 and toward the previously described rate control assembly 198. As before, retainer 144b normally holds shaft 144 in the retracted position.

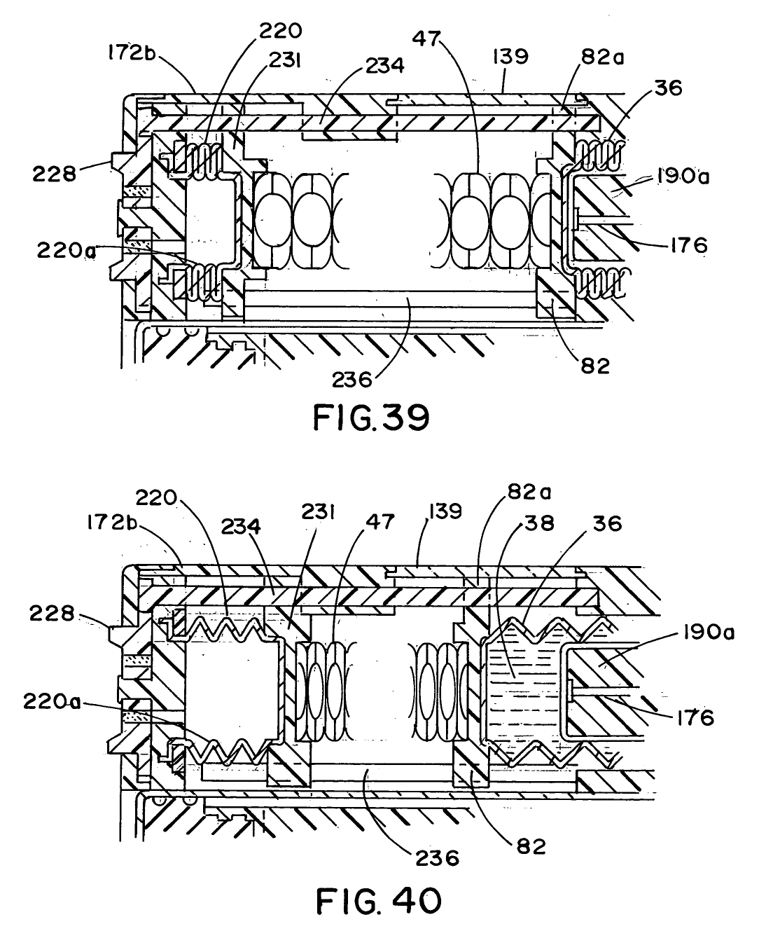

Considering next the important modulating means of this latest form of the invention for modulating the force exerted upon inner expandable housing 36 by the stored energy means, or spring 47. The modulating means in this latest form of the invention is similar in construction and operation to that previously described and here comprises a second expandable housing 220 that is carried by outer housing 172. Second expandable housing 220, which is operably associated with first expandable housing 36, comprises a bellows structure having an accordion like sidewall 220a that defines a fluid chamber 223 for containing a fluid such as air. Second expandable housing 220, which has an outlet 225 for permitting the flow of air there through, is movable from the substantially expanded configuration shown in FIG. 28 to the substantially collapsed configuration shown in FIG. 39, by a force exerted thereon by spring member 47.

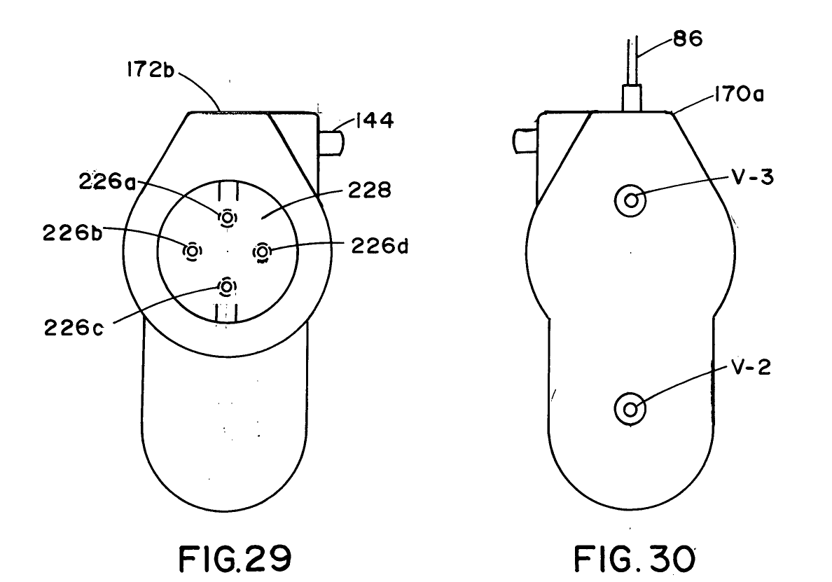

The modulating means of the present form of the invention further includes impedance means, here provided as a plurality of circumferentially spaced impedance frits 226a, 226b, 226c and 226d which are mounted within a control knob 228 that is rotatably carried proximate back of the drive by housing portion 172b (see FIGS. 29 and 32). These impedance frits, which can be constructed with different porosity, can be moved into index with bellows outlet 225 by controllably rotating control knob 228. In this way, the rate at which the fluid, such as air, will flow from reservoir 223 of bellows 220 to atmosphere via a selected frit can be controllably varied.

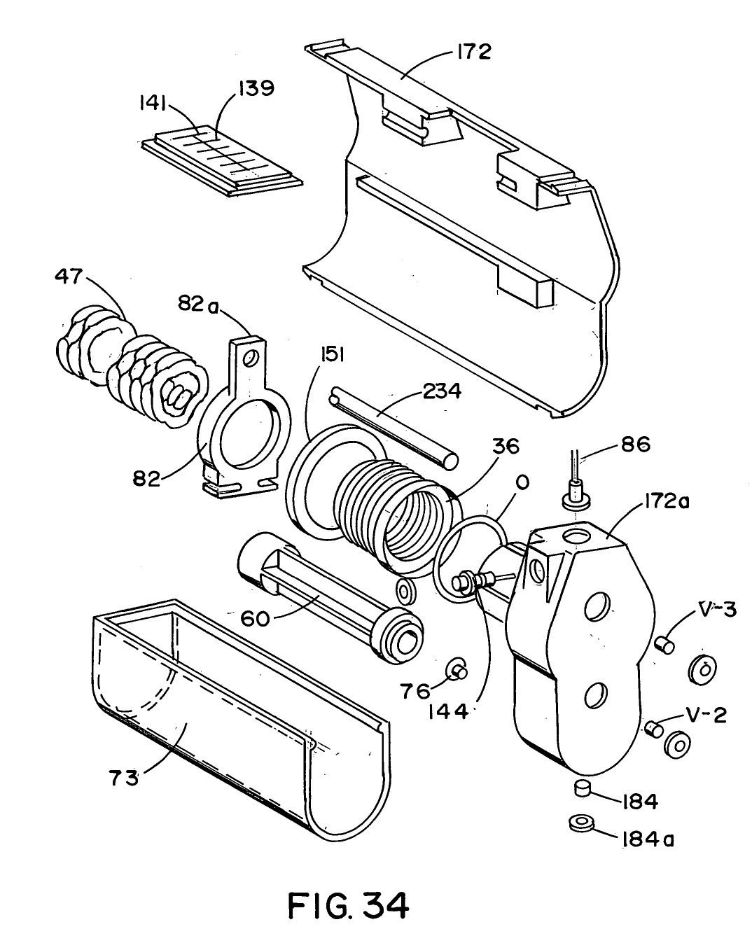

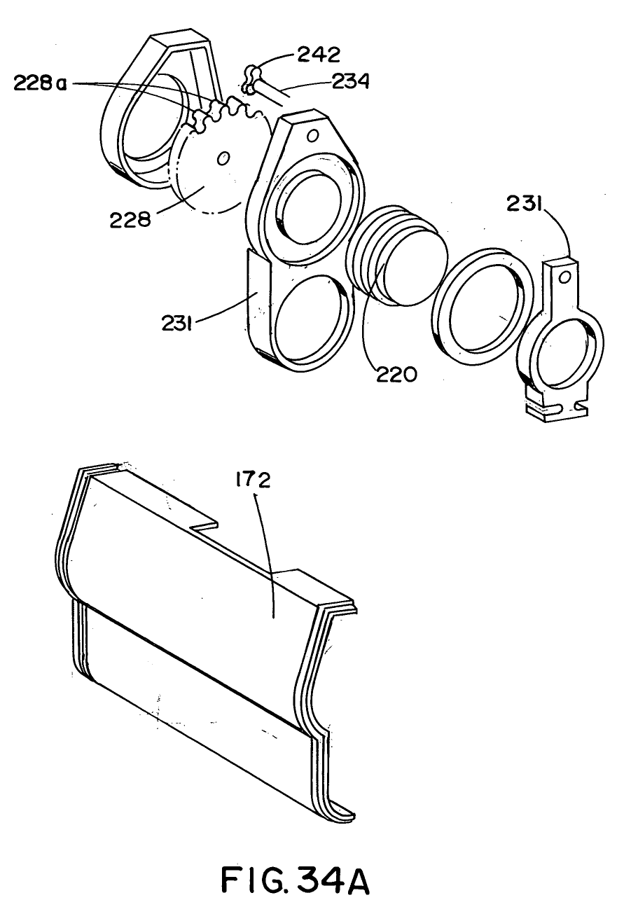

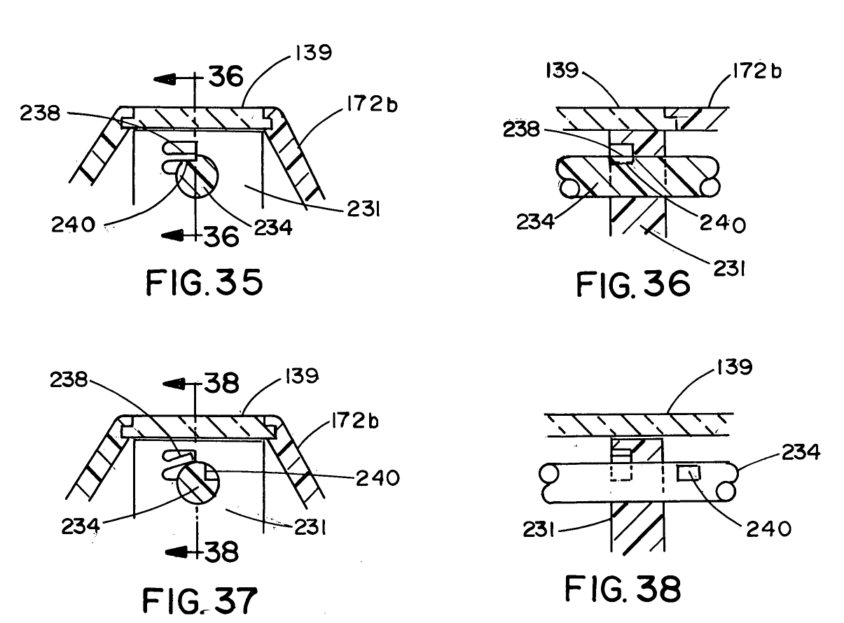

Disposed between spring 47 and second bellows housing 220 is an air collar 231 that is slidably movable within housing 172 along longitudinally extending shafts 234 and 236 (see FIGS. 28, 35 and 36). During the medicament delivery step, spring 47 acts upon indicator member 82, which, in turn, acts upon first bellows assembly 36 tending to collapse it and to cause the medicinal fluid contained within reservoir 38 to be forced outwardly thereof via reservoir outlet 178. At the same time, spring 47 acts upon an air collar 231 which, in turn, acts upon second bellows 220 tending to collapse it. However, before air collar 231 can slidably move along control shafts 234 and 236, the air collar must be released from its normally locked position shown in FIGS. 35 and 36 of the drawings. As indicated in FIGS. 35 and 36, sliding movement of air collar 231 is normally prevented by locking means shown here as a stop tab 238 that engages a shoulder 240 formed on rod 234. At the commencement of the medicament delivery step, control rod 234 is rotated by rotating the rearwardly mounted control knob 228. As illustrated in FIG. 34, control knob 228 is provided with a plurality of driving teeth 228a that engage driven teeth 242 provided proximate the end of control rod 234. With this construction, rotation of control knob 228 causes rotation of control shaft 234 which, in turn, causes the stop tab 238 to ride up on the shaft and out of locking engagement with shoulder 240 in the manner shown in FIG. 37 thereby allowing the air collar to move rearwardly of the control shaft in the manner shown in FIG. 38.

Rearward movement of the air collar due to the urging of spring 47, as illustrated in FIG. 28, will cause the air within chamber 223 of the second bellows assembly 220 to controllably flow through the selected porous frit that is in index with outlet 225 and then outwardly to atmosphere via the selected frit. As earlier described herein and as illustrated by FIG. 16A of the drawings, as the air collar moves rearwardly of the housing, the force being exerted on first bellows 36 by spring 47 will be modulated. As before, this modulation of the force exerted by spring 47 on second bellows 220 uniquely results in a more linear flow of medicinal fluid outwardly of the device as depicted by the lower graph of FIG. 16A.

Referring once again to FIGS. 45 and 46, the various types of springs suitable for use as the stored energy source of the invention are there illustrated and described. By way of background, springs are unlike other machine/structure components in that they undergo significant deformation when loaded—their compliance enables them to store readily recoverable mechanical energy.

With respect to the specific spring configurations shown in FIGS. 45 and 46, the following discussion amplifies the descriptive notations in these drawings.

Compression Springs:

Compression springs are open-wound helical springs that exert a load or force when compressed. They may be conical or taper springs, barrel or convex, concave or standard cylindrical in shape. The ends can be closed and ground, closed but unground, open and unground and supplied in alternate lengths. They also can include a configuration where a second compression spring of similar or different performance characteristics which can be installed inside the inside diameter of their first compression spring, i. e., a spring in a spring.

Many types of materials can be used in the manufacture with compression springs including: Commercial Wire (BS5216 HS3), Music Stainless Steel, Phosphur Bronze, Chrome Vanadium, Monel 400, Inconel 600, Inconel X750, Nimonic 90: Round wire, Square and Rectangular sections are also available. Exotic metals and their alloys with special properties can also be used for special and applications; they include such materials as beryllium copper, beryllium nickel, niobium, tantalum and titanium.

Compression springs can also be made from plastic including all thermoplastic materials used by custom spring winding service providers. Plastic springs may be used in light-to-medium duty applications for quiet and corrosion-resistant qualities.

Wave Spring:

Multiwave compression springs, an example of which is shown as “F” in FIG. 19B are readily commercially available from sources, such as the Smalley Company of Lake Zurich, Ill. As previously discussed, such springs operate as load bearing devices. They can take up play and compensate for dimensional variations within assemblies. A virtually unlimited range of forces can be produced whereby loads built either gradually or abruptly to reach a predetermined working height. This establishes a precise spring rate in which load if proportional to deflection, and can be turned to a particular load requirement.

Typically, a wave spring will occupy an extremely small area for the amount of work it performs. The use of this product is demanded, but not limited to tight axial and radial space restraints; one or more disc springs can be used and also of alternate individual thicknesses. Alternate embodiments of the basic disc spring design in a stacked assembly can be also utilized including specialty disc spring similar to the Belleville configuration called K Disc Springs manufactured by Adolf Schnorr BM8H of Singelfingen, Germany, as well as others manufactured by Christian Bauer GMBH of Welzheim, Germany.

Disc Springs:

Disc springs, examples of which are shown in G through P in FIGS. 45 and 46 comprise conically shaped annular discs (some with slotted or fingered configuration) which when loaded in the axial direction, change shape. In comparison to other types of springs, disc springs product small spring deflections under high loads. Some examples of the disc-shaped compression springs include a single or multiple stacked Belleville washer configuration as shown in G and H of FIG. 45, and depending on the requirements of the design (flow rate over time including bolus opportunity) one or more disc springs can be used and also of alternate individual thicknesses. Alternate embodiments of the basic disc spring design in a stacked assembly can be also utilized including specialty disc springs similar to the Belleville configuration called K disc springs manufactured by Adolf Schnorr GM8H of Singelfingen, Germany, as well as others manufactured by Christian Bauer GMBH of Welzheim, Germany.

Disc springs combine high energy storage capacity with low space requirement and uniform annular loading. They can provide linear or nonlinear spring loadings with their unique ability to combine high or low forces with either high or low deflection rates. They can be preloaded and under partial compression in the design application.

All these attributes, and more, come from single-component assemblies whose nontangle features (when compared to wirewound, compression springs) make them ideal for automatic assembly procedures.

With respect to the various springs discussed in the preceding paragraphs, it is to be understood that many alternate materials can be used in the design and application of disc springs and include carbon steel, chrome vanadium steel, stainless steel, heat resistant steels, and other special alloys such as nimonic, inconel, and beryllium copper. In some special applications, plastic disc springs designs can be used.

It should be further observed that, in comparison to other types of springs, disc springs produce small spring deflections under high loads. The ability to assemble disc springs into disc spring stacks overcomes this particular limitation. When disc springs are arranged in parallel (or nested), the load increases proportionate to the number of springs in parallel, while when disc springs are arranges in series (alternately) the travel will increase in proportion to the number of springs serially arranged. These assembly methods may be combined in use.

One special feature of the disc spring is, undoubtedly, the fact that the load/deflection characteristic curve can be designed to produce a wide variety of possibilities. In addition to practically linear load/deflection characteristic curves, regressive characteristics can be achieved and even disc springs which exhibit increasing spring deflection while the corresponding disc spring load is decreasing are readily available.

Slotted disc springs present a completely different case. Slotting changes the load/deflection characteristic of the single disc spring, providing larger spring deflections for greatly reduced loads. The slotted part is actually functioning as a series of miniature cantilever arms. In some cases the stacked, slotted disc spring, as shown in the clover dome design, will also produce a non-linear, stress strain curve with a noticed flat region (force/deflection). Application and use of this type of spring operating in this region will provide a near constant force between 15% and 75% of compression.

Having now described the invention in detail in accordance with the requirements of the patent statutes, those skilled in this art will have no difficulty in making changes and modifications in the individual parts or their relative assembly in order to meet specific requirements or conditions. Such changes and modifications may be made without departing from the scope and spirit of the invention, as set forth in the following claims.

1. A dispensing apparatus for dispensing fluids to a patient comprising:
(a) an outer housing;
(b) a first expandable housing disposed within said outer housing, said first expandable housing having a fluid reservoir provided with an inlet for permitting fluid flow into said fluid reservoir and an outlet for permitting fluid flow from said fluid reservoir;
(c) stored energy means disposed within said outer housing for exerting a force upon said first expandable housing to cause the fluid contained within said fluid reservoir to controllably flow through said outlet, said stored energy means comprising a compressively deformable, elastomeric member carried within said outer housing said, elastomeric member being expandable to cause fluid flow from said fluid reservoir;
(d) fill means carried by said outer housing for filling said reservoir with the fluid to be dispensed;
(e) modulating means carried by said outer housing for modulating the force exerted upon said first expandable housing by said stored energy means, said modulating means comprising a second expandable housing carried by said outer housing and operably associated with said first expandable housing; and
(f) dispensing means carried by said outer housing for dispensing fluid to the patient.

2. The apparatus as defined in claim 1 in which said elastomeric member comprises a yieldably deformable spring.

3. The apparatus as defined in claim 1 in which said first expandable housing comprises a bellows structure having an accordion-like side wall and, said bellows structure being movable from a substantially collapsed configuration to a substantially expanded configuration by fluid flowing into said fluid reservoir.

4. The apparatus as defined in claim 1 further including flow control means connected to said outer housing for controlling fluid flow between said reservoir and said dispensing means, said flow control means comprising a flow control member in fluid communication with said reservoir, said flow control member having a plurality of elongated flow control channels.

5. The apparatus as defined in claim 1 in which said fill means comprises a first fill vial receivable within said third portion of said outer housing.

6. The apparatus as defined in claim 1 in which said second expandable housing comprises a bellows structure having an accordion like side wall defining a fluid chamber for containing a fluid, said second expandable housing having a fluid outlet and being movable from a substantially expanded configuration to a substantially collapsed configuration by force exerted thereon by said stored energy means.

7. The apparatus as defined in claim 6 in which said modulating means further includes impedance means disposed within said fluid outlet of said second expandable housing for controllably impeding the flow of the fluid contained within said fluid chamber outwardly thereof.

8. The apparatus as defined in claim 7 in which said impedance means comprises a porous frit.

9. The apparatus as defined in claim 8 in which said fluid contained within said bellows structure comprises a gas.

10. A dispensing apparatus for dispensing fluids to a patient comprising:
(a) an outer housing having first, second and third portions;
(b) a first expandable housing disposed within said outer housing, said first expandable housing having a fluid reservoir provided with an inlet for permitting fluid flow into said fluid reservoir and an outlet for permitting fluid flow from said fluid reservoir, said first expandable housing comprising a bellows structure having an accordion-like side wall movable from a substantially collapsed configuration to a substantially expanded configuration by fluid flowing into said fluid reservoir;
(c) stored energy means disposed within said second portion of said outer housing for exerting a force upon said inner expandable housing to cause the fluid contained within said fluid reservoir to controllably flow through said outlet, said stored energy means comprising a compressively deformable, spring member carried within said outer housing, said spring member being expandable to cause fluid flow from said fluid reservoir;
(d) fill means carried by said outer housing for filling said reservoir with the fluid to be dispensed;
(e) modulating means carried by said outer housing for modulating the force exerted upon said inner expandable housing by said stored energy means, said modulating means comprising a second expandable housing carried by said outer housing and operably associated with said first expandable housing, said second expandable housing comprising a bellows structure having an accordion-like side wall defining an air chamber for containing air, said second expandable housing having an outlet for permitting the flow of air there through and being movable from a substantially expanded configuration to a substantially collapsed configuration by force exerted thereon by said spring member;
(f) dispensing means carried by said outer housing for dispensing fluid to the patient; and
(g) flow control means connected to said outer housing for controlling fluid flow between said reservoir and said dispensing means.

11. The apparatus as defined in claim 10 in which said fill means comprises a first fill vial receivable within said third portion of said outer housing and in which said third portion of said outer housing includes:
(a) a fluid passageway;
(b) a first chamber for telescopically receiving said first fill vial; and
(c) an elongated support mounted within said first chamber, said elongated support having an elongated hollow needle, said hollow needle defining a flow passageway in communication with said fluid passageway.

12. The apparatus as defined in claim 10 in which said third portion of said outer housing includes a cavity in communication with said inlet of said fluid reservoir and in which said fill means comprises a pierceable septum disposed within said cavity.

13. The apparatus as defined in claim 10 in which said modulating means further includes impedance means disposed within said outlet of said second expandable housing for controllably impeding the flow of the air contained within said air chamber outwardly thereof.

14. The apparatus as defined in claim 10 in which said flow control means comprises a flow control assembly including:
(a) an inlet manifold having an inlet port in communication of with said outlet of said first expandable housing; and
(b) an outlet manifold connected to said inlet manifold, said outlet manifold having an elongated micro channel in communication with said inlet port of said inlet manifold and in communication with said dispensing means.

15. The apparatus as defined in claim 10 in which said flow control means comprises a flow control assembly including:
(i) an ullage-defining member having a first portion disposed within said first expandable housing and a second portion having a fluid passageway in communication with said outlet of said fluid reservoir;
(ii) a flow control member rotatably mounted within said first portion of said ullage defining member, said flow control member having a plurality of elongated flow control channels, each of said plurality of elongated flow control channels having an inlet and an outlet; and
(iii) selector means rotatably connected to said second portion of said ullage defining member for rotating said flow control member to selectively align an outlet of one of said elongated flow control channels with said fluid passageway in said second portion of said ullage defining member.

16. The apparatus as defined in claim 15 in which said flow control assembly further comprises:
(a) an outer casing circumscribing said flow control member; and
(b) distribution means formed in said flow control member for distributing fluid from said fluid reservoir to each of said plurality of elongated flow control channels.

17. The apparatus as defined in claim 16, in which said flow control member is provided with an inlet passageway in communication with said fluid reservoir and in which said flow control assembly further includes filter means carried by said flow control member for filtering fluid flowing toward said distribution means.

18. The apparatus as defined in claim 17 in which said distribution means comprises a plurality of radially extending flow passageways formed in said flow control member.

19. The apparatus as defined in claim 18 in which said selector means comprises a selector knob connected to said flow control member, said selector knob having finger gripping means for imparting rotation to said selector knob to align said outlet of a selected one of said elongated flow control channels with said outlet of said fluid passageway in said second portion of said ullage defining member.

20. The apparatus as defined in claim 19, further including volume indicator means for indicating the volume of fluid remaining in said fluid reservoir.

21. The apparatus as defined in claim 20 further including disabling means for preventing fluid flow toward said dispensing means.

22. A dispensing apparatus for dispensing fluids to a patient comprising:
(a) an outer housing;
(b) a first expandable housing disposed within said outer housing, said first expandable housing having a fluid reservoir provided with an inlet for permitting fluid flow into said fluid reservoir and an outlet for permitting fluid flow from said fluid reservoir, said first expandable housing comprising a bellows structure having an accordion-like side wall movable from a substantially collapsed configuration to a substantially expanded configuration by fluid flowing into said fluid reservoir;
(c) stored energy means disposed within said outer housing for exerting a force upon said first expandable housing to cause the fluid contained within said fluid reservoir to controllably flow through said outlet, said stored energy means comprising a compressively deformable, wave spring carried within said outer housing, said wave spring being expandable to cause fluid flow from said fluid reservoir;
(d) fill means carried by said outer housing for filling said reservoir with the fluid to be dispensed;
(e) modulating means carried by said outer housing for modulating the force exerted upon said inner expandable housing by said stored energy means, said modulating means comprising:
(i) a second expandable housing carried by said outer housing and operably associated with said first expandable housing, said second expandable housing comprising a bellows structure having an accordion-like side wall defining an air chamber for containing air, said second expandable housing having an outlet for permitting the flow of air there through and being movable from a substantially expanded configuration to a substantially collapsed configuration by force exerted thereon by said spring member; and
(ii) impedance means disposed within said outlet of said expandable housing for controllably impeding the flow of the air contained within said air chamber outwardly thereof.
(f) dispensing means carried by said outer housing for dispensing fluid to the patient;
(g) flow control means connected to said outer housing for controlling fluid flow between said reservoir and said dispensing means;
(h) volume indicator means carried by said outer housing for indicating the volume of fluid remaining in said fluid reservoir; and
(i) disabling means carried by said outer housing for preventing fluid flow toward said dispensing means.

23. The apparatus as defined in claim 22 in which said fill means comprises a first fill vial receivable within said third portion of said outer housing and in which said third portion of said outer housing includes:
(a) a fluid passageway;
(b) a first chamber for telescopically receiving said first fill vial; and
(c) an elongated support mounted within said first chamber, said elongated support having an elongated hollow needle, said hollow needle defining a flow passageway in communication with said fluid passageway.

24. The apparatus as defined in claim 22 in which said outer housing includes a cavity in communication with said inlet of said fluid reservoir and in which said fill means comprises a pierceable septum disposed within said cavity.

25. The apparatus as defined in claim 22 in which said flow control means comprising a flow control assembly including:
(i) an ullage-defining member having a first portion disposed within said first expandable housing and a second portion having a fluid passageway in communication with said outlet of said fluid reservoir;
(ii) a flow control member rotatably mounted within said first portion of said ullage defining member, said flow control member having a plurality of elongated flow control channels, each of said plurality of elongated flow control channels having an inlet and an outlet; and
(iii) selector means rotatably connected to said second portion of said ullage defining member for rotating said flow control member to selectively align an outlet of one of said elongated flow control channels with said with fluid passageway in said second portion of said ullage defining member.

26. The apparatus as defined in claim 25 in which said flow control assembly further comprises:
(a) an outer casing circumscribing said flow control member; and
(b) distribution means formed in said flow control member for distributing fluid from said fluid reservoir to each of said plurality of elongated flow control channels.

27. The apparatus as defined in claim 26, in which said flow control member is provided with an inlet passageway in communication with said fluid reservoir and in which said flow control assembly further includes filter means carried by said flow control member for filtering fluid flowing toward said distribution means.

28. The apparatus as defined in claim 27 in which said distribution means comprises a plurality of radially extending flow passageways formed in said flow control member.

29. The apparatus as defined in claim 28 in which said selector means comprises a selector knob connected to said flow control member, said selector knob having finger gripping means for imparting rotation to said selector knob to align said outlet of a selected one of said elongated flow control channels with said outlet of said fluid passageway in said second portion of said ullage defining member.
